SEQUENCE LISTING (178 sequences)

SEQ ID NO: 1 - protein, 42 aa, synthetic construct
  REGION 1..42: note = Antimicrobial peptide with reference to North American bullfrog, Rana (Lithobates) catesbeiana
MFTMKKSLLLLFFLGTISLSLCEQERNADDDQGEVIEQKVKR

SEQ ID NO: 2 - protein, 29 aa, synthetic construct
  REGION 1..29: note = Antimicrobial peptide based on North American bullfrog, Rana (Lithobates) catesbeiana
AFLSTVKNTLTNVAGTMIDTFKCKITGVC

SEQ ID NO: 3 - protein, 31 aa, synthetic construct
  REGION 1..31: note = Antimicrobial peptide based on North American bullfrog, Rana (Lithobates) catesbeiana
VLLYLIITVSFPRRDANDEDGGEVTKEVVKR

SEQ ID NO: 4 - protein, 19 aa, synthetic construct
  REGION 1..19: note = Antimicrobial peptide based on North American bullfrog, Rana (Lithobates) catesbeiana
SLSGCWTKSFPRKPCLRNR

SEQ ID NO: 5 - protein, 36 aa, synthetic construct
  REGION 1..36: note = Antimicrobial peptide based on North American bullfrog, Rana (Lithobates) catesbeiana
MSSFCEITNVALTISLSSPRRGADEEEGNGEKEIKR

SEQ ID NO: 6 - protein, 25 aa, synthetic construct
  REGION 1..25: note = Antimicrobial peptide based on North American bullfrog, Rana (Lithobates) catesbeiana
SMLSVLKNLGKVGLGFVACKINKQC

SEQ ID NO: 7 - protein, 43 aa, synthetic construct
  REGION 1..43: note = Antimicrobial peptide based on North American bullfrog, Rana (Lithobates) catesbeiana
MTQSTQKWFKTKYWRVRNRPAMSPDLNPIEHLWRDLKKVVGKR

SEQ ID NO: 8 - protein, 33 aa, synthetic construct
  REGION 1..33: note = Antimicrobial peptide based on North American bullfrog, Rana (Lithobates) catesbeiana
NPSNLRALEELVKEECSEIPVERCKKLIYGYRK

SEQ ID NO: 9 - protein, 26 aa, synthetic construct
  REGION 1..26: note = Antimicrobial peptide based on North American bullfrog, Rana (Lithobates) catesbeiana
MRKRMTMRRMMKKKKSEKERRERGKR

SEQ ID NO: 10 - protein, 25 aa, synthetic construct
  REGION 1..25: note = Antimicrobial peptide based on North American bullfrog, Rana (Lithobates) catesbeiana
MMRVMRRKTKVIWEKKDFIGLYSID

SEQ ID NO: 11 - protein, 19 aa, synthetic construct
  REGION 1..19: note = Antimicrobial peptide based on North American bullfrog, Rana (Lithobates) catesbeiana
MFFMSSPRRDADEVKEVKR

SEQ ID NO: 12 - protein, 32 aa, synthetic construct
  REGION 1..32: note = Antimicrobial peptide based on North American bullfrog, Rana (Lithobates) catesbeiana
GFLDIIKNLGKTFAGHMLDKIKCTIGTCPPSP

SEQ ID NO: 13 - protein, 23 aa, synthetic construct
  REGION 1..23: note = Antimicrobial peptide based on North American bullfrog, Rana (Lithobates) catesbeiana
MITVSSPRRDADGDEGEVEEVKR

SEQ ID NO: 14 - protein, 31 aa, synthetic construct
  REGION 1..31: note = Antimicrobial peptide based on North American bullfrog, Rana (Lithobates) catesbeiana
GFLDIIKDTGKEFAVKILNNLKCKLAGGCPP

SEQ ID NO: 15 - protein, 71 aa, synthetic construct
  REGION 1..71: note = Putative AMP precursor based on Pseudacris regilla Ranatuerin-2PRc
MFTMKKSLLLFFFLGTISLSLCEEERDADDDQGEVVKKEVKRAFFTTVKNLVTNVAGTVIDKMKCKLTGQC

SEQ ID NO: 16 - protein, 71 aa, synthetic construct
  REGION 1..71: note = Putative AMP precursor based on R. catesbeiana HP2
MFTMKKSLLLLFFLGTISLSLCEQERNADDDQGEVIEQKVKRAFLSTVKNTLTNVAGTMIDTFKCKITGVC

SEQ ID NO: 17 - protein, 66 aa, synthetic construct
  REGION 1..66: note = Ranatuerin precursor sequences
MFTLKKSLLLLFFLGTITLSLCEQERGADEEEGNGEKEIKRSMLSVLKNLGKVGLGFVACKINKQC

SEQ ID NO: 18 - protein, 61 aa, synthetic construct
  REGION 1..61: note = Ranatuerin precursor sequence
MSSFCEITNVALTISLSSPRRGADEEEGNGEKEIKRSMLSVLKNLGKVGLGFVACKINKQC

SEQ ID NO: 19 - protein, 51 aa, synthetic construct
  REGION 1..51: note = Ranatuerin precursor sequence
MFFMSSPRRDADEVKEVKRGFLDIIKNLGKTFAGHMLDKIKCTIGTCPPSP

SEQ ID NO: 20 - protein, 68 aa, synthetic construct
  REGION 1..68: note = Ranatuerin precursor sequence
MFTMKKSLLLLFFLGTISLSLCEPQRDADEVKEVKRGFLDIIKNLGKTFAGHMLDKIKCTIGTCPPSP

SEQ ID NO: 21 - protein, 70 aa, synthetic construct
  REGION 1..70: note = Ranatuerin precursor sequence
MFTLKKSLLLLFFLGTINLSLCEEERDAEEERRDNPDERDVEVEKRFLPFIARLAAKVFPSIICSVTKKC

SEQ ID NO: 22 - protein, 71 aa, synthetic construct; identical to SEQ ID NO: 16
  REGION 1..71: note = Ranatuerin precursor sequence

SEQ ID NO: 23 - protein, 74 aa, synthetic construct
  REGION 1..74: note = Ranatuerin precursor sequence
MFTLKKSLLLLFFLGTITLSLCEQERGADEDNGGEMTEEEVKRGLFLDTLKGAAKDVAGKLLEGLKCKITGCKP

SEQ ID NO: 24 - protein, 67 aa, synthetic construct
  REGION 1..67: note = Ranacyclin precursor sequence from R. catesbeiana
MFTMKKSLLLLFFLGIISLSLCEQERDANDEEDGGEVTKEVVKRSLRGCWTKSFPPQPCLGKRLNMN

SEQ ID NO: 25 - protein, 63 aa, synthetic construct
  REGION 1..63: note = Ranacyclin precursor sequence from R. catesbeiana
MFTLKKSLLLLFFFGIISLSFCEQERDANDEEDGGEVTKEVVKRSLRGCWTKSYPPQPCLGKR

SEQ ID NO: 26 - protein, 50 aa, synthetic construct
  REGION 1..50: note = Ranacyclin precursor sequence from R. catesbeiana
VLLYLIITVSFPRRDANDEDGGEVTKEVVKRSLSGCWTKSFPRKPCLRNR

SEQ ID NO: 27 - protein, 42 aa, synthetic construct
  REGION 1..42: note = Catesbeianin-1 precursor sequence from R. catesbeiana
MFTMKKSEKERRERGKRMMRVMRRKTKVIWEKKDFIGLYSID

SEQ ID NO: 28 - protein, 51 aa, synthetic construct
  REGION 1..51: note = Catesbeianin-1 precursor sequence from R. catesbeiana
MRKRMTMRRMMKKKKSEKERRERGKRMMRVMRRKTKVIWEKKDFIGLYSID

SEQ ID NO: 29 - protein, 71 aa, synthetic construct
  REGION 1..71: note = Palustrin-Ca precursor sequence from R. catesbeiana
MFTMKKSLLLLFFLGTISLSLCEQERDADGDEGEVEEVKRGFLDIIKDTGKEFAVKILNNLKCKLAGGCPP

SEQ ID NO: 30 - protein, 54 aa, synthetic construct
  REGION 1..54: note = Palustrin-Ca precursor sequence from R. catesbeiana
MITVSSPRRDADGDEGEVEEVKRGFLDIIKDTGKEFAVKILNNLKCKLAGGCPP

SEQ ID NO: 31 - protein, 179 aa, synthetic construct
  REGION 1..179: note = Amolops loloensis Cathelicidin-AL sequence
MGLSATLWFLMGVAAGSMASPLLQWSEDDISVMALYSTDYYNKVSGEDVLYGLQENNTEYITDEKSRFHQLSFPIQKTVCQKSDNALTDDCAFKEGGVVKSCTSYFFEEDDRDIIVVTCQSQDGHREHSRVRRSRRGRGGGRRGGSGGRGGRGGGGRSGAGSSIAGVGSRGGGGGRHYA

SEQ ID NO: 32 - protein, 181 aa, synthetic construct
  REGION 1..181: note = R. catesbeiana Cathelicidin-AL sequence that aligns with an Amolops loloensis Cathelicidin-AL sequence
MGLSATFWFLMGLAASSMASPLLQWSEDDAAVMALYSADHYNKVSGEDVLYGLLENDTEYITDEKSRFHQLSFPIQETVCQKSDNNAPTDDCAFKEGGVVKSCTSYFFEEDDRDIVVVNCQSQDSHREHSRVRRSRSGRGGGGRGGGGRGGSRGGSRSGSRSSIAGGGSRGGSRGGGTRYA

SEQ ID NO: 33 - protein, 81 aa, synthetic construct
  REGION 1..81: note = Nanorana parkeri predicted LEAP2 sequence
MIPQLRKWMAIFVMCIVLIHQLEGAPMNSNDGSKTALRLRRMTPFWRGLSLRPVGASCRDDTECLTRLCRNQRCSLKTFAD

SEQ ID NO: 34 - protein, 81 aa, synthetic construct
  REGION 1..81: note = R. catesbeiana LEAP2 sequence aligning with Nanorana parkeri predicted LEAP2 sequence
MTPQLRKWTAIFVICIVLIHQLEGAPMSNTAGSKTLLRLRRMTPFWRGLSLRPVGASCRDDTECLTRLCRKERCSLKTFAD

SEQ ID NO: 35 - protein, 12 aa, synthetic construct
  REGION 1..12: note = P1_CCH, 'natural' antimicrobial peptide
FYFPVSRKFGGK

SEQ ID NO: 36 - protein, 12 aa, synthetic construct
  REGION 1..12: note = P1_CCH_F9R_Y2P, 'synthetic' AMP
FPFPVSRKRGGK

SEQ ID NO: 37 - protein, 26 aa, synthetic construct
  REGION 1..26: note = P2_CCH, 'natural' AMP
FFPRVLPLANKFLPTIYCALPKSVGN

SEQ ID NO: 38 - protein, 26 aa, synthetic construct
  REGION 1..26: note = P2_CCH_T15K_P7R, 'synthetic' AMP
FFPRVLRLANKFLPKIYCALPKSVGN

SEQ ID NO: 39 - protein, 32 aa, synthetic construct
  REGION 1..32: note = P3_CCH, 'natural' AMP
GLLDIIKDTGKTTGILMDTLKCQMTGRCPPSS

SEQ ID NO: 40 - protein, 32 aa, synthetic construct
  REGION 1..32: note = P3_CCH_D8K_Q23C, 'synthetic' AMP
GLLDIIKKTGKTTGILMDTLKCCMTGRCPPSS

SEQ ID NO: 41 - protein, 31 aa, synthetic construct
  REGION 1..31: note = P4_CCH, 'natural' AMP
GLLDIIKTTGKDFAVKILDNLKCKLAGGCPP

SEQ ID NO: 42 - protein, 31 aa, synthetic construct
  REGION 1..31: note = P4_CCH_P31K, 'synthetic' AMP
GLLDIIKTTGKDFAVKILDNLKCKLAGGCPK

SEQ ID NO: 43 - protein, 24 aa, synthetic construct
  REGION 1..24: note = P5_CCH, 'natural' AMP
FFPIIARLAAKVIPSLVCAVTKKC

SEQ ID NO: 44 - protein, 24 aa, synthetic construct
  REGION 1..24: note = P5_CCH_A19K, 'synthetic' AMP
FFPIIARLAAKVIPSLVCKVTKKC

SEQ ID NO: 45 - protein, 31 aa, synthetic construct
  REGION 1..31: note = P6_CCH, 'natural' AMP
GLWETIKTTGKSIALNLLDKIKCKIAGGCPP

SEQ ID NO: 46 - protein, 31 aa, synthetic construct
  REGION 1..31: note = P6_CCH_S12K, 'synthetic' AMP
GLWETIKTTGKKIALNLLDKIKCKIAGGCPP

SEQ ID NO: 47 - protein, 26 aa, synthetic construct
  REGION 1..26: note = P7_CCH, 'natural' AMP
ATAWRIPPPGMQPIIPIRIRPLCGKQ

SEQ ID NO: 48 - protein, 26 aa, synthetic construct
  REGION 1..26: note = P7_CCH_P9R_R5M, 'synthetic' AMP
ATAWMIPPRGMQPIIPIRIRPLCGKQ

SEQ ID NO: 49 - protein, 12 aa, synthetic construct
  REGION 1..12: note = P8_CCH, 'natural' AMP
FPAIICKVSKNC

SEQ ID NO: 50 - protein, 12 aa, synthetic construct
  REGION 1..12: note = P8_CCH_N11K, 'synthetic' AMP
FPAIICKVSKKC

SEQ ID NO: 51 - protein, 15 aa, synthetic construct
  REGION 1..15: note = P9_CCH_2, 'synthetic' AMP
FLTKPGMTFGKLLGK

SEQ ID NO: 52 - protein, 15 aa, synthetic construct
  REGION 1..15: note = P10_CCH, 'natural' AMP
SNRDFFKVNIFRLCG

SEQ ID NO: 53 - protein, 15 aa, synthetic construct
  REGION 1..15: note = P10_CCH_2, 'synthetic' AMP
SNRKFFKVRIFRLCG

SEQ ID NO: 54 - protein, 25 aa, synthetic construct
  REGION 1..25: note = P11_CCH, 'natural' AMP
ALVAKIQKFPVFNTLKLCKLELEII

SEQ ID NO: 55 - protein, 25 aa, synthetic construct
  REGION 1..25: note = P11_CCH_E21K_E23R, 'synthetic' AMP
ALVAKIQKFPVFNTLKLCKLKLRII

SEQ ID NO: 56 - protein, 13 aa, synthetic construct
  REGION 1..13: note = P12_CCH, 'natural' AMP
IAGQVAAAKQKHI

SEQ ID NO: 57 - protein, 13 aa, synthetic construct
  REGION 1..13: note = P12_CCH2, 'synthetic' AMP
IAGQKARAKQKHI

SEQ ID NO: 58 - protein, 20 aa, synthetic construct
  REGION 1..20: note = P13_CCH, 'natural' AMP
IQRLPVINMLKLWKLELEII

SEQ ID NO: 59 - protein, 20 aa, synthetic construct
  REGION 1..20: note = P13_CCH_N8K_E18K, 'synthetic' AMP
IQRLPVIKMLKLWKLELKII

SEQ ID NO: 60 - protein, 21 aa, synthetic construct
  REGION 1..21: note = P14_CCH, 'natural' AMP
IQRLPVIVILPSLYCVICRTC

SEQ ID NO: 61 - protein, 21 aa, synthetic construct
  REGION 1..21: note = P14_CCH_2, 'synthetic' AMP
IQRLPVIVILPSLYCVICRKK

SEQ ID NO: 62 - protein, 35 aa, synthetic construct
  REGION 1..35: note = P15_CCH, 'natural' AMP
LRCPTPHYNFENGIGNHLMWNIIWLNAQQMSYKNK

SEQ ID NO: 63 - protein, 35 aa, synthetic construct
  REGION 1..35: note = P15_CCH_2, 'synthetic' AMP
LRCPTPHYRFENGIGNHLMWNIIWLNAQQMSYCNK

SEQ ID NO: 64 - protein, 25 aa, synthetic construct
  REGION 1..25: note = P16_CCH, 'natural' AMP
SNRDFFMVNIFGLCGPFGIMERKRR

SEQ ID NO: 65 - protein, 25 aa, synthetic construct
  REGION 1..25: note = P16_CCH_2, 'synthetic' AMP
SNRKFFMVNIFGLCGPFGIMKRKRR

SEQ ID NO: 66 - protein, 12 aa, synthetic construct
  REGION 1..12: note = P1 variant AMP sequence
  REGION 1..9: note = MISC_FEATURE - Xaa is any amino acid
XXXPXXXXXGGK

SEQ ID NO: 67 - protein, 12 aa, synthetic construct
  REGION 1..12: note = Group P1 variant AMP sequence
  REGION 5..9: note = MISC_FEATURE - Xaa is any amino acid
FYFPXXXXXGGK

SEQ ID NO: 68 - protein, 12 aa, synthetic construct
  REGION 1..12: note = Group P1 variant AMP sequence
  REGION 1..12: note = MISC_FEATURE - Xaa is any amino acid
XYFXXSRKXXXX

SEQ ID NO: 69 - protein, 12 aa, synthetic construct
  REGION 1..12: note = Group P1 variant AMP sequence
  REGION 2..12: note = MISC_FEATURE - Xaa is any amino acid
FXFXVSRKXXXX

SEQ ID NO: 70 - protein, 12 aa, synthetic construct
  REGION 1..12: note = Group P1 variant AMP sequence
  REGION 1..11: note = MISC_FEATURE - Xaa is any amino acid
XYFXXXXKFXXK

SEQ ID NO: 71 - protein, 12 aa, synthetic construct
  REGION 1..12: note = Group P1 variant AMP sequence
  REGION 1..8: note = MISC_FEATURE - Xaa is any amino acid
XXXXXXXXFGGK

SEQ ID NO: 72 - protein, 12 aa, synthetic construct
  REGION 1..12: note = Group P1 variant AMP sequence
  REGION 2..12: note = MISC_FEATURE - Xaa is any amino acid
FYXPVXRXFXXX

SEQ ID NO: 73 - protein, 26 aa, synthetic construct
  REGION 1..26: note = Group P2 variant AMP sequence
  REGION 5..22: note = MISC_FEATURE - Xaa is any amino acid
FFPRXXXXXXXFLPTXXXXXXXSVGN

SEQ ID NO: 74 - protein, 26 aa, synthetic construct
  REGION 1..26: note = Group P2 variant AMP sequence
  REGION 1..26: note = MISC_FEATURE - Xaa is any amino acid
XXXXVLPLANKXXXXIYCXXXXXXXX

SEQ ID NO: 75 - protein, 26 aa, synthetic construct
  REGION 1..26: note = Group P2 variant AMP sequence
  REGION 4..23: note = MISC_FEATURE - Xaa is any amino acid
FFPXXLPLANXXLPXXXXXLPXXVGN

SEQ ID NO: 76 - protein, 26 aa, synthetic construct
  REGION 1..26: note = Group P2 variant AMP sequence
  REGION 1..26: note = MISC_FEATURE - Xaa is any amino acid
XXPRVLXXXXXXXXXXXXXXPKSXXX

SEQ ID NO: 77 - protein, 26 aa, synthetic construct
  REGION 1..26: note = Group P2 variant AMP sequence
  REGION 2..26: note = MISC_FEATURE - Xaa is any amino acid
FXXXXXXLANKXXXTIYCXXXXSVXX

SEQ ID NO: 78 - protein, 26 aa, synthetic construct
  REGION 1..26: note = Group P2 variant AMP sequence
  REGION 3..25: note = MISC_FEATURE - Xaa is any amino acid
FFXXVLPXXXXXLXTXYCALPKXVXN

SEQ ID NO: 79 - protein, 26 aa, synthetic construct
  REGION 1..26: note = Group P2 variant AMP sequence
  REGION 1..26: note = MISC_FEATURE - Xaa is any amino acid
XXXXXXXLANXFXPXIXXALPKXXGX

SEQ ID NO: 80 - protein, 32 aa, synthetic construct
  REGION 1..32: note = Group P3 variant AMP sequence
  REGION 4..27: note = MISC_FEATURE - Xaa is any amino acid
GLLXXXXXXXXXXXXXXXXXXXXXXXXCPPSS

SEQ ID NO: 81 - protein, 32 aa, synthetic construct
  REGION 1..32: note = Group P3 variant AMP sequence
  REGION 1..31: note = MISC_FEATURE - Xaa is any amino acid
XXXXXXKXXXKXXGXLMXXXXXXMXGXXPPXS

SEQ ID NO: 82 - protein, 32 aa, synthetic construct
  REGION 1..32: note = Group P3 variant AMP sequence
  REGION 4..32: note = MISC_FEATURE - Xaa is any amino acid
GLLXIIXXXGXTTGILMXXLXXXMXGXXPPXX

SEQ ID NO: 83 - protein, 32 aa, synthetic construct
  REGION 1..32: note = Group P3 variant AMP sequence
  REGION 1..30: note = MISC_FEATURE - Xaa is any amino acid
XLXXXXXXXXKXXXXXXXTLKCQMTXXCXXSS

SEQ ID NO: 84 - protein, 32 aa, synthetic construct
  REGION 1..32: note = Group P3 variant AMP sequence
  REGION 4..32: note = MISC_FEATURE - Xaa is any amino acid
GLLXIIKXTGKTTGILMXXLKXXXXGXXXXXX

SEQ ID NO: 85 - protein, 32 aa, synthetic construct
  REGION 1..32: note = Group P3 variant AMP sequence
  REGION 2..28: note = MISC_FEATURE - Xaa is any amino acid
GXLXIIKXTGXXTXIXMXTLKCQXTGRXPPSS

SEQ ID NO: 86 - protein, 32 aa, synthetic construct
  REGION 1..32: note = Group P3 variant AMP sequence
  REGION 4..32: note = MISC_FEATURE - Xaa is any amino acid
GLLXXXKXTGKXTXIXXXTLKXQXTGRXXXXX

SEQ ID NO: 87 - protein, 32 aa, synthetic construct
  REGION 1..32: note = Group P3 variant AMP sequence
  REGION 1..32: note = MISC_FEATURE - Xaa is any amino acid
XXXXIIXXTXXTXGXLXXTXXCQXTXRXXXXX

SEQ ID NO: 88 - protein, 31 aa, synthetic construct
  REGION 1..31: note = Group P4 variant AMP sequence
  REGION 4..31: note = MISC_FEATURE - Xaa is any amino acid
GLLXIIXXXGXXXXXXILXXLXXXLAGGXXX

SEQ ID NO: 89 - protein, 31 aa, synthetic construct
  REGION 1..31: note = Group P4 variant AMP sequence
  REGION 4..29: note = MISC_FEATURE - Xaa is any amino acid
GLLXXXKTTGKXFAVKILXNLXXXXXXXXPP

SEQ ID NO: 90 - protein, 31 aa, synthetic construct
  REGION 1..31: note = Group P4 variant AMP sequence
  REGION 1..31: note = MISC_FEATURE - Xaa is any amino acid
XXXXIIKTXXXXFAVXXXXXXKCKLAGGXXX

SEQ ID NO: 91 - protein, 31 aa, synthetic construct
  REGION 1..31: note = Group P4 variant AMP sequence
  REGION 1..28: note = MISC_FEATURE - Xaa is any amino acid
XXXXIIXXXXKXXXXKXXXNLKCKXXXXCPP

SEQ ID NO: 92 - protein, 31 aa, synthetic construct
  REGION 1..31: note = Group P4 variant AMP sequence
  REGION 4..31: note = MISC_FEATURE - Xaa is any amino acid
GLLXXXKTTGXXXXXXXLXNLKCXXAXXCXX

SEQ ID NO: 93 - protein, 31 aa, synthetic construct
  REGION 1..31: note = Group P4 variant AMP sequence
  REGION 4..29: note = MISC_FEATURE - Xaa is any amino acid
GLLXXXKXXXXXFAVXXLXXLXXXXXXXXPP

SEQ ID NO: 94 - protein, 24 aa, synthetic construct
  REGION 1..24: note = Group P5 variant AMP sequence
  REGION 1..21: note = MISC_FEATURE - Xaa is any amino acid
XXXXXXXLAAKXXXSLVXXXXKKC

SEQ ID NO: 95 - protein, 24 aa, synthetic construct
  REGION 1..24: note = Group P5 variant AMP sequence
  REGION 1..24: note = MISC_FEATURE - Xaa is any amino acid
XFPIIAXLAAXVIPXLVXAVTXXX

SEQ ID NO: 96 - protein, 24 aa, synthetic construct
  REGION 1..24: note = Group P5 variant AMP sequence
  REGION 4..24: note = MISC_FEATURE - Xaa is any amino acid
FFPXXAXXXXKXXPXXXXXXXXXX

SEQ ID NO: 97 - protein, 24 aa, synthetic construct
  REGION 1..24: note = Group P5 variant AMP sequence
  REGION 1..24: note = MISC_FEATURE - Xaa is any amino acid
XXXXXXXLAAXVIPXLXXXXTXXX

SEQ ID NO: 98 - protein, 24 aa, synthetic construct
  REGION 1..24: note = Group P5 variant AMP sequence
  REGION 7..16: note = MISC_FEATURE - Xaa is any amino acid
FFPIIAXXXXXXXXXXVCAVTKKC

SEQ ID NO: 99 - protein, 24 aa, synthetic construct
  REGION 1..24: note = Group P5 variant AMP sequence
  REGION 1..19: note = MISC_FEATURE - Xaa is any amino acid
XXXIIXRXXXKVIXSXVCXVTKKC

SEQ ID NO: 100 - protein, 24 aa, synthetic construct
  REGION 1..24: note = Group P5 variant AMP sequence
  REGION 11..24: note = MISC_FEATURE - Xaa is any amino acid
FFPIIARLAAXVIXSLXCAVXXXX

SEQ ID NO: 101 - protein, 31 aa, synthetic construct
  REGION 1..31: note = Group P6 variant AMP sequence
  REGION 8..26: note = MISC_FEATURE - Xaa is any amino acid
GLWETIKXXXKXXXXXXXXKXXXXXXGGCPP

SEQ ID NO: 102 - protein, 31 aa, synthetic construct
  REGION 1..31: note = Group P6 variant AMP sequence
  REGION 1..31: note = MISC_FEATURE - Xaa is any amino acid
XXXXXXXTTGXXXXXXXXXXXKCKXXXXCXX

SEQ ID NO: 103 - protein, 31 aa, synthetic construct
  REGION 1..31: note = Group P6 variant AMP sequence
  REGION 1..29: note = MISC_FEATURE - Xaa is any amino acid
XXXXTIXXXGXXIALXLLXXIXXXIAXXXPP

SEQ ID NO: 104 - protein, 31 aa, synthetic construct
  REGION 1..31: note = Group P6 variant AMP sequence
  REGION 6..29: note = MISC_FEATURE - Xaa is any amino acid
GLWETXKTTXXSXXLNLLDKIXXKIAXXXPP

SEQ ID NO: 105 - protein, 31 aa, synthetic construct
  REGION 1..31: note = Group P6 variant AMP sequence
  REGION 1..31: note = MISC_FEATURE - Xaa is any amino acid
XXXXXIKXXGKSIALXXXXKXKXKXXGGXXX

SEQ ID NO: 106 - protein, 31 aa, synthetic construct
  REGION 1..31: note = Group P6 variant AMP sequence
  REGION 1..31: note = MISC_FEATURE - Xaa is any amino acid
XXXXXXXXXXKSIAXNLLXXIXCXIAGGXXX

SEQ ID NO: 107 - protein, 26 aa, synthetic construct
  REGION 1..26: note = Group P7 variant AMP sequence
  REGION 5..26: note = MISC_FEATURE - Xaa is any amino acid
ATAWXIXXXGMXXIIXIXIXXLXGXX

SEQ ID NO: 108 - protein, 26 aa, synthetic construct
  REGION 1..26: note = Group P7 variant AMP sequence
  REGION 1..22: note = MISC_FEATURE - Xaa is any amino acid
XXXWXIXXXGMQXXXXXXXXXXCGKQ

SEQ ID NO: 109 - protein, 26 aa, synthetic construct
  REGION 1..26: note = Group P7 variant AMP sequence
  REGION 1..26: note = MISC_FEATURE - Xaa is any amino acid
XXXXXXPPPXXQPXXPXXXXPXXXXX

SEQ ID NO: 110 - protein, 26 aa, synthetic construct
  REGION 1..26: note = Group P7 variant AMP sequence
  REGION 4..24: note = MISC_FEATURE - Xaa is any amino acid
ATAXXXPPPXXXPXXPXXXXPXCXKQ

SEQ ID NO: 111 - protein, 26 aa, synthetic construct
  REGION 1..26: note = Group P7 variant AMP sequence
  REGION 1..26: note = MISC_FEATURE - Xaa is any amino acid
XXXWXIXXXGMXXXXXIXIXXLXGXX

SEQ ID NO: 112 - protein, 26 aa, synthetic construct
  REGION 1..26: note = Group P7 variant AMP sequence
  REGION 1..26: note = MISC_FEATURE - Xaa is any amino acid
XXXXXXXXXXXXPXXPXXIXXLXGXX

SEQ ID NO: 113 - protein, 26 aa, synthetic construct
  REGION 1..26: note = Group P7 variant AMP sequence
  REGION 4..24: note = MISC_FEATURE - Xaa is any amino acid
ATAXRXXXXXXQXIIXIRIRXLCXKQ

SEQ ID NO: 114 - protein, 12 aa, synthetic construct
  REGION 1..12: note = Group P8 variant AMP sequence
  REGION 6..12: note = MISC_FEATURE - Xaa is any amino acid
FPAIIXXXXXXX

SEQ ID NO: 115 - protein, 12 aa, synthetic construct
  REGION 1..12: note = Group P8 variant AMP sequence
  REGION 2..11: note = MISC_FEATURE - Xaa is any amino acid
FXXXXCXXXKXC

SEQ ID NO: 116 - protein, 12 aa, synthetic construct
  REGION 1..12: note = Group P8 variant AMP sequence
  REGION 1..12: note = MISC_FEATURE - Xaa is any amino acid
XXAIIXXVSKXX

SEQ ID NO: 117 - protein, 12 aa, synthetic construct
  REGION 1..12: note = Group P8 variant AMP sequence
  REGION 1..12: note = MISC_FEATURE - Xaa is any amino acid
XPAXXCKXXXXX

SEQ ID NO: 118 - protein, 12 aa, synthetic construct
  REGION 1..12: note = Group P8 variant AMP sequence
  REGION 3..7: note = MISC_FEATURE - Xaa is any amino acid
FPXXXXXVSKNC

SEQ ID NO: 119 - protein, 12 aa, synthetic construct
  REGION 1..12: note = Group P8 variant AMP sequence
  REGION 1..12: note = MISC_FEATURE - Xaa is any amino acid
XXXIICKVSXNX

SEQ ID NO: 120 - protein, 15 aa, synthetic construct
  REGION 1..15: note = Group P9 variant AMP sequence
  REGION 5..15: note = MISC_FEATURE - Xaa is any amino acid
FLTFXGXXFGXXXGX

SEQ ID NO: 121 - protein, 15 aa, synthetic construct
  REGION 1..15: note = Group P9 variant AMP sequence
  REGION 1..15: note = MISC_FEATURE - Xaa is any amino acid
XXXXPGMXFXXLLXX

SEQ ID NO: 122 - protein, 15 aa, synthetic construct
  REGION 1..15: note = Group P9 variant AMP sequence
  REGION 1..14: note = MISC_FEATURE - Xaa is any amino acid
XXXXPGMXXXKXXXK

SEQ ID NO: 123 - protein, 15 aa, synthetic construct
  REGION 1..15: note = Group P9 variant AMP sequence
  REGION 5..15: note = MISC_FEATURE - Xaa is any amino acid
FLTFXXXXXXXLLGX

SEQ ID NO: 124 - protein, 15 aa, synthetic construct
  REGION 1..15: note = Group P9 variant AMP sequence
  REGION 2..14: note = MISC_FEATURE - Xaa is any amino acid
FXXFXXXTFGKXXXK

SEQ ID NO: 125 - protein, 15 aa, synthetic construct
  REGION 1..15: note = Group P9 variant AMP sequence
  REGION 1..7: note = MISC_FEATURE - Xaa is any amino acid
XLTXXXXTFGKLLGK

SEQ ID NO: 126 - protein, 15 aa, synthetic construct
  REGION 1..15: note = Group P10 variant AMP sequence
  REGION 1..15: note = MISC_FEATURE - Xaa is any amino acid
XXXXFFXVNIFXLXX

SEQ ID NO: 127 - protein, 15 aa, synthetic construct
  REGION 1..15: note = Group P10 variant AMP sequence
  REGION 3..15: note = MISC_FEATURE - Xaa is any amino acid
SNXXXXXVXXXRXXX

SEQ ID NO: 128 - protein, 15 aa, synthetic construct
  REGION 1..15: note = Group P10 variant AMP sequence
  REGION 1..12: note = MISC_FEATURE - Xaa is any amino acid
XXXXFFXXXIFXLCG

SEQ ID NO: 129 - protein, 15 aa, synthetic construct
  REGION 1..15: note = Group P10 variant AMP sequence
  REGION 3..12: note = MISC_FEATURE - Xaa is any amino acid
SNXXXXKXXXXXLCG

SEQ ID NO: 130 - protein, 15 aa, synthetic construct
  REGION 1..15: note = Group P10 variant AMP sequence
  REGION 1..15: note = MISC_FEATURE - Xaa is any amino acid
XXRXXXKVNIFXXCX

SEQ ID NO: 131 - protein, 15 aa, synthetic construct
  REGION 1..15: note = Group P10 variant AMP sequence
  REGION 2..14: note = MISC_FEATURE - Xaa is any amino acid
SXXXFFXVXIXXXXG

SEQ ID NO: 132 - protein, 15 aa, synthetic construct
  REGION 1..15: note = Group P10 variant AMP sequence
  REGION 2..15: note = MISC_FEATURE - Xaa is any amino acid
SXRDFFKXNXXRXCX

SEQ ID NO: 133 - protein, 25 aa, synthetic construct
  REGION 1..25: note = Group P11 variant AMP sequence
  REGION 1..25: note = MISC_FEATURE - Xaa is any amino acid
XXXXXIQKXXXXNTLKXXKXXLXXX

SEQ ID NO: 134 - protein, 25 aa, synthetic construct
  REGION 1..25: note = Group P11 variant AMP sequence
  REGION 6..25: note = MISC_FEATURE - Xaa is any amino acid
ALVAKXXXFPVFXXXXLCXLXXXXX

SEQ ID NO: 135 - protein, 25 aa, synthetic construct
  REGION 1..25: note = Group P11 variant AMP sequence
  REGION 9..23: note = MISC_FEATURE - Xaa is any amino acid
ALVAKIQKXXXXXXXXXXKLXXXII

SEQ ID NO: 136 - protein, 25 aa, synthetic construct
  REGION 1..25: note = Group P11 variant AMP sequence
  REGION 1..25: note = MISC_FEATURE - Xaa is any amino acid
XXXXXXXKXPXXNTLKXCKXEXEXX

SEQ ID NO: 137 - protein, 25 aa, synthetic construct
  REGION 1..25: note = Group P11 variant AMP sequence
  REGION 1..25: note = MISC_FEATURE - Xaa is any amino acid
XXXXXXQXFXVFXTLKLXKLXLXXX

SEQ ID NO: 138 - protein, 25 aa, synthetic construct
  REGION 1..25: note = Group P11 variant AMP sequence
  REGION 1..23: note = MISC_FEATURE - Xaa is any amino acid
XLVAKIXXXPVXNXXXLXXXXLXII

SEQ ID NO: 139 - protein, 25 aa, synthetic construct
  REGION 1..25: note = Group P11 variant AMP sequence
  REGION 2..22: note = MISC_FEATURE - Xaa is any amino acid
AXXAXIXXFXXFXXXXXCXXEXEII

SEQ ID NO: 140 - protein, 13 aa, synthetic construct
  REGION 1..13: note = Group P12 variant AMP sequence
  REGION 1..13: note = MISC_FEATURE - Xaa is any amino acid
XXGQVXXXKXKXX

SEQ ID NO: 141 - protein, 13 aa, synthetic construct
  REGION 1..13: note = Group P12 variant AMP sequence
  REGION 3..13: note = MISC_FEATURE - Xaa is any amino acid
IAXXXAAAXXXXX

SEQ ID NO: 142 - protein, 13 aa, synthetic construct
  REGION 1..13: note = Group P12 variant AMP sequence
  REGION 1..10: note = MISC_FEATURE - Xaa is any amino acid
XXGXXXXXKXKHI

SEQ ID NO: 143 - protein, 13 aa, synthetic construct
  REGION 1..13: note = Group P12 variant AMP sequence
  REGION 2..8: note = MISC_FEATURE - Xaa is any amino acid
IXXQVXXXKQKHI

SEQ ID NO: 144 - protein, 13 aa, synthetic construct
  REGION 1..13: note = Group P12 variant AMP sequence
  REGION 1..11: note = MISC_FEATURE - Xaa is any amino acid
XXXQVXXXXQXHI

SEQ ID NO: 145 - protein, 20 aa, synthetic construct
  REGION 1..20: note = Group P13 variant AMP sequence
  REGION 1..20: note = MISC_FEATURE - Xaa is any amino acid
XXRXPXXXXXKLWKXXLXXX

SEQ ID NO: 146 - protein, 20 aa, synthetic construct
  REGION 1..20: note = Group P13 variant AMP sequence
  REGION 3..18: note = MISC_FEATURE - Xaa is any amino acid
IQXXXVXXXLXXXXLXXXII

SEQ ID NO: 147 - protein, 20 aa, synthetic construct
  REGION 1..20: note = Group P13 variant AMP sequence
  REGION 1..20: note = MISC_FEATURE - Xaa is any amino acid
XXXLXXXNMXXXWKXXXXXX

SEQ ID NO: 148 - protein, 20 aa, synthetic construct
  REGION 1..20: note = Group P13 variant AMP sequence
  REGION 1..20: note = MISC_FEATURE - Xaa is any amino acid
XXXLPXINMXKLXXXXLXXX

SEQ ID NO: 149 - protein, 21 aa, synthetic construct
  REGION 1..21: note = Group P14 variant AMP sequence
  REGION 5..17: note = MISC_FEATURE - Xaa is any amino acid
IQRLXXXXXXXSLYXXXCRTC

SEQ ID NO: 150 - protein, 21 aa, synthetic construct
  REGION 1..21: note = Group P14 variant AMP sequence
  REGION 1..21: note = MISC_FEATURE - Xaa is any amino acid
XXXLPVXVXLPSLYXXXXXXX

SEQ ID NO: 151 - protein, 21 aa, synthetic construct
  REGION 1..21: note = Group P14 variant AMP sequence
  REGION 4..21: note = MISC_FEATURE - Xaa is any amino acid
IQRXXXIVIXXXXXCVIXXXX

SEQ ID NO: 152 - protein, 21 aa, synthetic construct
  REGION 1..21: note = Group P14 variant AMP sequence
  REGION 1..17: note = MISC_FEATURE - Xaa is any amino acid
XXXXPVXXXXPSLYXXXCRTC

SEQ ID NO: 153 - protein, 21 aa, synthetic construct
  REGION 1..21: note = Group P14 variant AMP sequence
  REGION 5..20: note = MISC_FEATURE - Xaa is any amino acid
IQRLXVIXILXXXXCXXCXXC

SEQ ID NO: 154 - protein, 21 aa, synthetic construct
  REGION 1..21: note = Group P14 variant AMP sequence
  REGION 1..21: note = MISC_FEATURE - Xaa is any amino acid
XXXLPXXXXXPXXXXVIXXTX

SEQ ID NO: 155 - protein, 21 aa, synthetic construct
  REGION 1..21: note = Group P14 variant AMP sequence
  REGION 1..11: note = MISC_FEATURE - Xaa is any amino acid
XXXLXVIVILXSLYCVICRTC

SEQ ID NO: 156 - protein, 35 aa, synthetic construct
  REGION 1..35: note = Group P15 variant AMP sequence
  REGION 2..35: note = MISC_FEATURE - Xaa is any amino acid
LXXPXPXYXFXXGIGXXXXWXXXWLNAQQMXXXXX

SEQ ID NO: 157 - protein, 35 aa, synthetic construct
  REGION 1..35: note = Group P15 variant AMP sequence
  REGION 1..35: note = MISC_FEATURE - Xaa is any amino acid
XXCPTPXXXFXXXXXNHLXXXIIWLXXXXMXXXXX

SEQ ID NO: 158 - protein, 35 aa, synthetic construct
  REGION 1..35: note = Group P15 variant AMP sequence
  REGION 4..30: note = MISC_FEATURE - Xaa is any amino acid
LRCXXXXXXXENGXXXXXMWNXXXXXXXXXSYKNK

SEQ ID NO: 159 - protein, 35 aa, synthetic construct
  REGION 1..35: note = Group P15 variant AMP sequence
  REGION 1..34: note = MISC_FEATURE - Xaa is any amino acid
XXXXTXHYNXENGIGNHLMXNXXXXNXQXXXXKXK

SEQ ID NO: 160 - protein, 35 aa, synthetic construct
  REGION 1..35: note = Group P15 variant AMP sequence
  REGION 2..35: note = MISC_FEATURE - Xaa is any amino acid
LXCXXXHXNXXXXXGNHXXWXXXWLXXXXMSXXNX

SEQ ID NO: 161 - protein, 35 aa, synthetic construct
  REGION 1..35: note = Group P15 variant AMP sequence
  REGION 1..35: note = MISC_FEATURE - Xaa is any amino acid
XRXPXPHXXFXXXXXXXXXXXIIXXXAXQXSYXXX

SEQ ID NO: 162 - protein, 25 aa, synthetic construct
  REGION 1..25: note = Group P16 variant AMP sequence
  REGION 4..25: note = MISC_FEATURE - Xaa is any amino acid
SNRXXXMXXXXGLXGPXXIMXXXXX

SEQ ID NO: 163 - protein, 25 aa, synthetic construct
  REGION 1..25: note = Group P16 variant AMP sequence
  REGION 1..25: note = MISC_FEATURE - Xaa is any amino acid
XXXXFFMXXXXXXCXXFGXXXXKXX

SEQ ID NO: 164 - protein, 25 aa, synthetic construct
  REGION 1..25: note = Group P16 variant AMP sequence
  REGION 5..21: note = MISC_FEATURE - Xaa is any amino acid
SNRDXXXXXIFGLXGPXXIMXRKRR

SEQ ID NO: 165 - protein, 25 aa, synthetic construct
  REGION 1..25: note = Group P16 variant AMP sequence
  REGION 2..25: note = MISC_FEATURE - Xaa is any amino acid
SXXXXXXVNXXXXCXXFGXXEXXXX

SEQ ID NO: 166 - protein, 25 aa, synthetic construct
  REGION 1..25: note = Group P16 variant AMP sequence
  REGION 1..23: note = MISC_FEATURE - Xaa is any amino acid
XXXXXXXVNIFXXXXPXXXXXRXRR

SEQ ID NO: 167 - protein, 71 aa, synthetic construct
  REGION 1..71: note = antimicrobial peptide
KSKLSLKKQGTIHLDAQSSCDVMHFPKCDLAPNVQRQAWLFKVASKEAKELRYYLLNPYLDVSARNVGSKV

SEQ ID NO: 168 - protein, 47 aa, synthetic construct
  REGION 1..47: note = Antimicribial peptide
KAGEGERGEREVLNHQKTILEPSSCPLISPHSTGLGHRPSLFRLTLA

SEQ ID NO: 169 - protein, 42 aa, synthetic construct
  REGION 1..42: note = Antimircrobial Peptide
LKGIKNAAQLLRFPPNCKLCSCTVFVHKDHCVVQEASGVFRF

SEQ ID NO: 170 - protein, 47 aa, synthetic construct
  REGION 1..47: note = Antimicrobial Peptide
NAARDHSATRCKQRSARLQIAAQDYRSQRSARLQIATQRKITDRNTA

SEQ ID NO: 171 - protein, 15 aa, synthetic construct
  REGION 1..15: note = Antimicrobial Peptide
LKPSNIQVKLQYIYW

SEQ ID NO: 172 - protein, 122 aa, synthetic construct
  REGION 1..122: note = Antimicrobial peptide precursor
MNCGSFPCDACDVCEYVDAKTKLKLPNGRWHSIQFRVTCQTPGVIYLAQCLCGGFYIGKTKRQFFKRIRDHIKPIRKNKMDTAISRHVGIHHNFNPQFIKFSALEHIPQTLAVAALIASCYN

SEQ ID NO: 173 - protein, 29 aa, synthetic construct
  REGION 1..29: note = Antimicrobial peptide precursor
MEEIVFPLQHPFHLDCLFFLLRHLSWEKT

SEQ ID NO: 174 - protein, 43 aa, synthetic construct
  REGION 1..43: note = Antimicrobial peptide precursor
MSIKKKEEMIQVKGMLKWKNDFYQLLERFSVLCLEKNPEMLKL

SEQ ID NO: 175 - protein, 35 aa, synthetic construct
  REGION 1..35: note = Antimicrobial peptide precursor
MIQVKGMLKWKNDFYQLLERFSVLCLEKNPEMLKL

SEQ ID NO: 176 - protein, 27 aa, synthetic construct
  REGION 1..27: note = Antimicrobial peptide precursor
MPKKKEETIQMKGMLKWKNDFFQLLHA

SEQ ID NO: 177 - protein, 41 aa, synthetic construct
  REGION 1..41: note = Antimicrobial peptide precursor
MSGSRIGLPLALFPVTFVKISLFILLSSSSSAFLLGEHSYC

SEQ ID NO: 178 - protein, 85 aa, synthetic construct
  REGION 1..85: note = Antimicrobial peptide precursor
MSSPRRDANEEERRDDPDERDVEVEKRLLPVTTSENVLVHRGGQKAGMDHREVTQGWREDLGHQEELSLNLQENNGGHPQFMPFQ

(19) United States
(12) Patent Application Publication
(10) Pub. No.: US 2025/0332215 A1
(43) Pub. Date: Oct. 30, 2025

(54) ANTIMICROBIAL PEPTIDES

(71) Applicants: PROVINCIAL HEALTH SERVICES AUTHORITY, Vancouver (CA); UVIC INDUSTRY PARTNERSHIPS INC., Victoria (CA)

(72) Inventors: Inanc BIROL, Vancouver (CA); Caren C. HELBING, Victoria (CA); Stewart Austin HAMMOND, Saskatoon (CA)

(21) Appl. No.: 19/088,030

(22) Filed: Mar. 24, 2025

Related U.S. Application Data
(63) Continuation of application No. 17/312,806, filed on Jun. 10, 2021.
(60) Provisional application No. 62/778,450, filed on Dec. 12, 2018.

(51) Int. Cl. A61K 38/17; A61P 31/04; G01N 33/50; G01N 33/569; G16B 15/30

(52) U.S. Cl. CPC A61K 38/1703; A61P 31/04; G01N 33/5011; G01N 33/56911; G16B 15/30; G01N 2500/04

(57) ABSTRACT

Antimicrobial peptides (AMPs), small compounds that often exhibit broad spectrum antimicrobial activity, are garnering interest as potential therapeutics against antibiotic-resistant bacterial pathogens. Development of new AMPs is arduous due to the practical limitations of classical protein-based discovery approaches. A high throughput bioinformatics approach is described which is able to confirm identification of known AMPs from the North American bullfrog (Rana (Lithobates) castebeiana) genome, and a bioinformatics approach is used to develop new AMPs, as described herein as SEQ ID NOs: 1-178. The described AMPs exhibit antimicrobial activity against Mycobacterium smegmatis via microtitre broth dilution assays, indicating broader efficacy.

### CROSS REFERENCE TO RELATED APPLICATIONS

This application claims the benefit of U.S. patent application Ser. No. 17/312,806 entitled “ANTIMICROBIAL PEPTIDES” filed Jun. 10, 2021, which is the U.S. National Phase of International Patent Application No. PCT/CA 2019/051778 filed Dec. 10, 2019 entitled “ANTIMICROBIAL PEPTIDES”, which claims priority to U.S. Provisional Patent Application No. 62/778,450 filed Dec. 12, 2018, the subject matter of each of which is incorporated by reference herein in its entirety.

### STATEMENT REGARDING FEDERALLY SPONSORED RESEARCH OR DEVELOPMENT

This invention was made in part with United States government support under Grant No. R01HG007182, awarded by the National Institutes of Health. The United States government has certain rights in the invention.

### SEQUENCE LISTING

The instant application contains a Sequence Listing which has been submitted electronically in XML format and is hereby incorporated by reference in its entirety. Said XML format Sequence Listing is named “PAT 107205AW-2—SEQ LIST.xml”, was created on Mar. 22, 2025, and is 263,400 bytes in size.

### FIELD

The present disclosure relates generally to antimicrobial peptides for the treatment or mitigation of disease.

### BACKGROUND

There is a need for peptides and pharmaceutical compositions thereof which are useful as therapies for microbial infections or as chemopreventive agents to slow or arrest the progression of microbial infections.

Use of antibiotics in livestock may have direct and indirect impact on medical use in addressing human disease. The ubiquitous use of antibiotics in all industries has contributed to the emergence of superbugs which have become resistant to the most common antibiotics. Some strains illustrate multi-drug resistance, which is a global concern. Although the search for new antibiotic approaches continues in earnest to address challenges in both human and animal health.

Consumers have concerns about the use of prophylactic antibiotics due to the potential environmental impact, increasing drug resistance, and the possible consumption of antibiotic lace meat or dairy products. Restrictions on prophylactic antibiotic use in livestock that have been implemented to address these concerns, but have downstream consequences such as increased rates of animal infections, leading to productivity loss due to the increase disease burden. Sick animals that are then treated with antibiotics will continue to contribute to potential drug resistance. Poultry and swine raised in close quarters are particularly susceptible to the rapid spread of disease. Different approaches to reducing infections disease in livestock animals are under development, including investigation of new antibiotic approaches, and development of vaccines. While small molecule drugs have conventionally been used, antimicrobial peptide and polypeptide therapeutic approaches are also under consideration.

It is, therefore, desirable to find new antimicrobial approaches to reduce the onset and spread of disease in humans and animals.

### SUMMARY

Peptides and/or amino acid sequences with antimicrobial properties are described herein. A bioinformatics approach, starting with sequences exhibiting effect, and making strategic modifications thereto, has led to the discovery of antimicrobial peptides. In a bioinformatics approach, sufficient similarity among sequences can be maintained so as to permit functional equivalency. Sequences similar to isolated sequences from which a consensus is derived are also described. Such similar sequences contain conserved amino acid substitutions and a limited number of non-conserved modifications.

It is an object of the present disclosure to provide antimicrobial peptides, which may obviate or mitigate at least one disadvantage of previous antimicrobial approaches.

There is described herein an antimicrobial peptide consisting of the amino acid sequence according to any one of SEQ ID NOs:1-178, or more particularly according to any one of SEQ ID NOs:1-45 and 47-178. Further, there is described herein an antimicrobial peptide comprising: an amino acid sequence according to any one of SEQ ID NO:1 to SEQ ID NO: 166, or a fragment or variant thereof, having at least 65% amino acid sequence identity to any one of SEQ ID NO:1 SEQ ID NO:166.

Further, there is described herein a composition comprising the described antimicrobial peptide together with a suitable excipient.

The composition comprising the described antimicrobial peptide may be a composition for use in in treatment or prevention of a disease or condition, such as infectious disease.

A use for the antimicrobial peptide is provided, for treatment or prevention of a disease or condition in a subject in need thereof. Further, the use of the antimicrobial peptide for preparation of a medicament for treatment or prevention of a disease or condition in a subject in need thereof is also described herein. Additionally, a method of treating or preventing a disease or condition is described, comprising administering to a subject in need thereof an effective amount of the antimicrobial peptide or composition thereof. The disease may be, for example, an infectious disease. The subject may be a human or an animal, such as a livestock animal or a companion animal.

A lipid vesicle comprising the antimicrobial peptide is described. A nucleic acid molecule encoding the antimicrobial peptide is also provided, as is a vector comprising such a nucleic acid molecule.

A method of identifying a target molecule associated with an infectious agent is described, in which the target molecule binds to the antimicrobial peptide. The method comprises the step of screening a library of candidate target molecules associated with the infectious agent, for a molecule that binds to the antimicrobial peptide. A kit for conducting such a method for identifying a target molecule associated with an infectious agent is also described, in which the kit comprises the antimicrobial peptide described herein together with instructions.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached FIGURES.

FIG. 1 shows Clustal omega alignments of putative AMP precursor sequences with their closest known AMP matches. Panel A compares Pseudacris regilla Ranatuerin-2PRc (SEQ ID NO:15) with the R. catesbeiana HP2 (SEQ ID NO:16). Panel B aligns seven Ranatuerin precursor sequences (SEQ ID NO:17 to SEQ ID NO:23). Panel C aligns three Ranacyclin precursor sequences from R. catesbeiana (SEQ ID NO:24 to SEQ ID NO:26). Panel D compares Catesbeianin-1 precursor sequences from R. catesbeiana (SEQ ID NO:27 and SEQ ID NO:28). Panel E aligns Palustrin-Ca precursor sequences from R. catesbeiana (SEQ ID NO:29 and SEQ ID NO:30).

FIG. 2 illustrates a SABLE secondary structure prediction comparisons between the derived mature peptides of Panel A-HP2; Panel B-HP3; and Panel C-HP5 versus known mature AMP sequences. Panel D provides a legend for the SABLE predictions with amino acid (AA) position indicated at the top, the predicted secondary structure in the middle and the relative solvent accessibility (RSA) at the bottom.

FIG. 3 illustrates that putative and known AMP-encoding transcripts show differential expression in R. catesbeiana premetamorphic tadpole back skin (black bars), liver (dark grey bars), olfactory epithelium (light grey bars), and tail fin (white bars).

FIG. 4 shows Clustal omega alignments of putative AMP precursor sequences with their closest known AMP matches. Panel A compares Amolops loloensis Cathelicidin-AL sequence (SEQ ID NO: 31) with the corresponding R. catesbeiana Cathelicidin-AL sequence (SEQ ID NO:32). Panel B compares Nanorana parkeri predicted LEAP2 sequence (SEQ ID NO:33) with the corresponding R. catesbeiana LEAP2 sequence (SEQ ID NO:34).

FIG. 5 shows RNA-seq data representing transcripts encoding the indicated putative and known AMPs are shown for tadpole Panel A—back skin; Panel B—liver; Panel C—olfactory epithelium; and Panel D tail fin from controls (black bars) versus tadpoles exposed to 10 nM T3 for 48 h (grey bars).

FIG. 6 shows ranatuerin-1 and ranatuerin-3RC genes containing 2 exons and which are alternatively spliced. The structure of the genes and derived transcripts encoding (Panel A) Ranatuerin-1 and Ranatuerin-1 (HP4), and (Panelo B) Ranatuerin-3RC and Ranatuerin-3RC (HP8), with top illustration representing the gene drawn to the indicated scale with the exonic portions depicted as black rectangles and intronic regions depicted by the thick black line. Intronic regions are shown as thin lines that are spliced out in the labelled transcripts below the gene. Grey rectangles in the spliced transcript indicate the untranslated regions, and hatched rectangles indicate the open reading frame.

FIG. 7 shows the ranatuerin-2PRc (HP2), ranatuerin-2RC, and ranatuerin-4 genes, having 3 exons. The structure of the genes and derived transcripts encoding (Panel A) Ranatuerin-2PRc (HP2), (Panel B) Ranatuerin-2RC, and (Panel C) Ranatuerin-4 are shown.

FIG. 8 shows two genes, one with 3 exons and the other with 1 exon, encoding Ranacyclins. The structure of the genes and derived transcripts encoding (Panel A) Ranacyclin-Ca and Ranacyclin-Cc, and (Panel B) Ranacyclin-Ca (HP3) are shown.

FIG. 9 shows Palustrin-Ca encoded by a 2 exon gene, and Palustrin-Ca (HP9) and HP5 are encoded by single exon genes. The structure of the genes and derived transcripts encoding (Panel A) Palustrin-Ca, (Panel B) Palustrin-Ca (HP9), and (Panel C) HP5 are shown.

FIG. 10 illustrates both LEAP2 and Cathelicidin-AL as derived from four exons. The structure of the genes and derived transcripts encoding R. catesbeiana (Panel A) LEAP2 and (Panel B) Cathelicidin-AL are shown.

### DETAILED DESCRIPTION

Peptides and/or amino acid sequences with antimicrobial properties are described herein. A bioinformatics approach, starting with sequences exhibiting effect, and making strategic modifications thereto, has led to the discovery of antimicrobial peptides. In a bioinformatics approach, sufficient similarity among sequences can be maintained so as to permit functional equivalency. Sequences similar to isolated sequences from which a consensus is derived are also described. Such similar sequences may contain conserved amino acid substitutions together with a limited number of non-conserved substitutions, such as modifications or deletions, but while still maintaining functionality.

These peptides and their pharmaceutical compositions and modifications thereof are also useful as therapies for microbial infections or as chemopreventative agents to slow or arrest the progression of microbial infections. Modifications of peptides described herein may include but are not limited to incorporation of the peptides or their modifications in lipid vesicles for enhanced therapeutic delivery and the modulation of other ADMET properties (absorption, distribution, metabolism, excretion, toxicity) as well.

Chemical modifications of the peptides are described, which are known to individuals skilled in the art of peptide chemistry to be useful to enhance stability and otherwise make the peptides more drug-like and useful for the desired applications. Such modifications include peptide cyclization and the use of amino acids of opposite chirality—so-called D-amino acids. Such modifications also include alternative backbone chemistries and novel side chains that retain the binding specificity.

Also described is the application of the peptides, and modifications of the peptides obvious to those skilled in the art, to other microbial targets. Antimicrobial therapies useful and effective in one type of infection may be useful and effective in other diseases.

Also described are vector constructs incorporating the disclosed peptides and/or their amino acid sequences and coding nucleic acid sequences for the purposes of the production of antimicrobial peptides.

The peptides described herein, and the modifications thereof are also useful in combination with other antimicrobial agents for the treatment or prevention of disease, such as an infectious disease or a cancer.

Uses of the AMPs either alone or as part of a kit in a procedure to isolate and identify their binding partners (target molecules) associated with the infectious agent are also described.

The peptides and/or amino acid sequences described herein have selective antimicrobial properties. Further aspects and advantages will become apparent from consideration of the ensuing description of various embodiments. A person skilled in the art will realize that other embodiments, combinations and variations are possible, and that the details described herein can be modified in a number of respects, all without departing from the overall concept. Thus, the following drawings, descriptions and examples are to be regarded as illustrative in nature and not restrictive.

Treatment or prevention of a disease or condition encompasses treatment before and after outward signs or symptoms of the disease or condition are present in the subject. For example, a subject exposed an infectious agent may or may not exhibit symptoms. Further, the prevention or prophylaxis of a disease or condition may encompass partial prevention, lessening of severity when onset occurs, decreasing likelihood of outward signs or symptoms, or preventing the spread of infection by keeping severity so low as to be undetectable or negligible. Treatment and prevention may involve modulating the immune system of the subject to the extent that the subject's own defenses ward off the disease or condition, such as infection. An inflammatory or anti-inflammatory effect of the peptides described herein may modulate the outward signs or symptoms of a disease or condition.

Anti-cancer activity, such as against solid tumours or liquid tumours, may be modulated by peptides as described herein. Indirect attack on cancer cells by the peptides described herein through effects on the immune system by the peptides may alleviate cancerous cell growth.

An antimicrobial peptide is described comprising: an amino acid sequence according to any one of SEQ ID NO:1 to SEQ ID NO:166, or a fragment or variant thereof, having at least 65% amino acid sequence identity to any one of SEQ ID NO:1 to SEQ ID NO:166. The threshold of amino acid sequence identity for the variant or fragment may optionally be at least 70%, at least 75%, at least 80%, at least 85%, at least 90%, or at least 95% amino acid sequence identity to any one of SEQ ID NO:1 to SEQ ID NO:166.

The antimicrobial peptide may be modified, or may be a variant which comprises a modification that is a conservative amino acid substitution. Such an amino acid sequences as are known in the art may include the following candidates, with the substitutable options shown in parentheses: Ala (Gly, Ser); Arg (Gly, GIn); Asn (GIn, His); Asp (Glu); Cys (Ser); Gln (Asn, Lys); Glu (Asp); Gly (Ala, Pro); His (Asn, Gln); Ile (Leu, Val); Leu (Ile, Val); Lys (Arg, GIn); Met (Leu, Ile); Phe (Met, Leu, Tyr); Ser (Thr, GIy); Thr (Ser; Val); Trp (Tyr); Tyr (Trp, Phe); and Val (Ile, Leu). Furthermore, ‘functional’ variants, mutations, insertions, or deletions encompass sequences in which the activity or function is substantially the same as that of the reference sequence from which the altered sequence is derived. Activity or function may be tested according to such parameters as described herein, such as MIC or MBC. Further, it may be desirable to reduce the antigenicity of a peptide, for example by PEGylated, or the peptide may comprise a D-amino acid. The peptide may be cyclized.

The antimicrobial peptide may be a peptide or a fragment that is up to 30 amino acids in length. For example, it may be a peptide or a fragment of up to 20 amino acids in length. An exemplary antimicrobial peptide may be one that comprises or consists of an amino acid sequence according to any one of SEQ ID NO:1 to SEQ ID NO:65.

A composition is described herein which comprises the antimicrobial peptide as described herein, together with a suitable excipient, such as a pharmaceutically acceptable carrier. The composition may be one that is suitable for use in treatment or prevention of a disease or condition, such as an infectious disease, or a cancer, such as may be attributable to a solid tumour or a liquid tumour.

The composition may be formulated for oral, injectable, rectal, topical, transdermal, nasal, or ocular delivery. Such compositions can thus be administered to subjects in need thereof through any acceptable route, such as topically (as by powders, ointments, or drops); oral tablets, capsules, gels or liquids; or rectal suppositories. Further modes of delivery include mucosally, sublingually, parenterally, intravaginally, intraperitoneally, bucally, ocularly, or intranasally.

When formulated for oral use or administration in a liquid formulation, the excipients or ingredients may include but are not limited to those accepted in the art of pharmaceutical formulations, for example emulsions, microemulsions, solutions, suspensions, syrups and elixirs. Liquid dosage forms may contain inert diluents such as water or other solvents, solubilizing agents, emulsifiers, ethyl alcohol, isopropyl alcohol, ethyl carbonate, ethyl acetate, benzyl alcohol, benzyl benzoate, propylene glycol, 1,3-butylene glycol, or dimethylformamide. Further, a liquid formulation may comprise oils such as cottonseed, groundnut, corn, germ, olive, castor, and sesame oils; glycerol, tetrahydrofurfuryl alcohol, polyethylene glycols and fatty acid esters of sorbitan; and mixtures thereof. Besides inert diluents, such oral compositions can also include adjuvants such as wetting agents, emulsifying and suspending agents, sweetening, flavoring, and perfuming agents.

The composition may be one that is lyophilized. The composition may comprise a suitable preservative.

The composition may be one that is distributed evenly in a diet intended for livestock, such as swine or poultry. Such a composition may be sprayed or mixed into a ground or powdered ingredient, and then mixed evenly into a coarser animal feed to ensure even distribution.

A use of the antimicrobial peptide is provided herein, for treatment or prevention of a disease or condition in a subject in need thereof, such as an infectious disease. The disease or condition may be a cancer, such as a solid tumour or a liquid tumour.

Further, a use is provided for preparation of a medicament for treatment or prevention of such a disease or condition in a subject in need thereof. A method of treating or preventing such a disease or condition is also described herein, which comprises administering to a subject in need thereof an effective amount of the peptide or the composition described herein.

The disease or condition may be one attributable to Gram-negative bacteria, or it may be a disease or condition attributable to Gram-positive bacteria. The disease or condition may be one that is attributable to acid fast bacteria, or one that is attributable to bacteria that has become resistant to other drugs. Such diseases or conditions may be ones attributable to E. coli, S. enterica, S. aureus, P. aeruginosa, S. pyogenes, M. smegmatis, MRSA, S. enteritidis or S. heidelberg bacteria, for example.

Further, the disease or condition may be a cancer, such as a solid tumour or a liquid tumour.

A lipid vesicle may be used to deliver the antimicrobial peptide described herein. A nucleic acid molecule encoding the antimicrobial peptide described is also envisioned. A vector comprising the nucleic acid molecule is also encompassed.

A method of identifying a target molecule associated with an infectious agent is described, wherein the target molecule binds to the antimicrobial peptide described herein. Such a method involves the step of screening a library of candidate target molecules associated with the infectious agent, for a molecule that binds to the antimicrobial peptide. The infectious agent may be Gram-negative bacteria, or may be Gram-positive bacteria. Further, the infections agent may be acid fast bacteria, or bacteria that has become resistant to other drugs. Exemplary infectious agents include but are not limited to E. coli, S. enterica, S. aureus, P. aeruginosa, S. pyogenes, M. smegmatis, MRSA, S. enteritidis or S. heidelberg bacteria. Further, a method of identifying a target molecule for modulating biological activity is described, wherein the target molecule binds to a peptide as described herein. The method comprising the step of screening a library of candidate target molecules for a molecule that binds to the peptide. Modulating of biological activity may comprise anti-tumour action, anti-inflammatory action, or inflammatory action. In such methods of target identification, the screening of a library of candidate target molecules may comprise in silico screening.

A kit is encompassed herein for identifying a target molecule associated with an infectious agent. Such a kit comprises an antimicrobial peptide as described herein together with instructions for conducting the method described herein for identifying a target molecule associated with the infectious agent. Optionally, additional reagents may be provided with the kit. A kit for identifying a target molecule for modulating biological activity, is also described. Such a kit comprises a peptide, as described herein, together with instructions for conducting a screening method for molecules that bind to the peptide.

### EXAMPLES

The following Examples outline exemplary embodiments and/or studies conducted pertaining thereto. While the Examples are illustrative, they should not be viewed as limiting.

### Example 1

Bioinformatics Approach: Antimicrobial Peptides from R. catesbeiana Transcripts

Summary Antimicrobial peptides (AMPs) exhibit broad-spectrum antimicrobial activity, and have promise as new therapeutic agents. While the adult North American bullfrog (Rana [Lithobates] catesbeiana) is a prolific source of high-potency AMPs, the aquatic tadpole represents a relatively untapped source for new AMP discovery. The recent publication of the bullfrog genome and transcriptomic resources provides an opportune bridge between known AMPs and bioinformatics-based AMP discovery. The objective of the present study was to identify novel AMPs with therapeutic potential using a combined bioinformatics and wet lab-based approach. In the present study, seven novel AMP precursor-encoding transcripts expressed in the tadpole were identified. Comparison of their amino acid sequences with known AMPs revealed evidence of mature peptide sequence conservation with variation in the prepro sequence. Two mature peptide sequences were unique and demonstrated bacteriostatic and bactericidal activity against Mycobacteria but not Gram-negative or Gram-positive bacteria. Nine known and seven novel AMP-encoding transcripts were detected in premetamorphic tadpole back skin, olfactory epithelium, liver, and/or tail fin. Treatment of tadpoles with 10 nM 3,5,3′-triiodothyronine for 48 h did not affect transcript abundance in the back skin, and had limited impact on these transcripts in the other three tissues. Gene mapping revealed considerable diversity in size (1.6-15 kbp) and exon number (one to four) of AMP-encoding genes with clear evidence of alternative splicing leading to both prepro and mature amino acid sequence diversity. These findings verify the accuracy and utility of the bullfrog genome assembly, and set a firm foundation for bioinformatics-based AMP discovery.

### Introduction

Antibiotic resistance among bacterial pathogens that cause prevalent global diseases has emerged as one of the most critical public threats facing the world today¹⁻³. A n analysis conducted by the Centers for Disease Control and Prevention estimates that at least 23,000 deaths in the United States each year are attributed to infections caused by antibiotic-resistant organisms¹. In 2015, the World Health Organization endorsed a global action plan to combat antimicrobial resistance with strategic objectives that include optimizing the use of antimicrobial agents and sustainable investment in countering antimicrobial resistance⁴. Consequently, discovery and development of alternative antimicrobials is an urgent global need. As an alternative to traditional antibiotic therapy, antimicrobial peptides (AMPs) are garnering interest as potential therapeutics⁵. AMPs are a diverse class of peptides produced by all multicellular organisms as a defense against a broad spectrum of pathogens including bacteria, fungi, and viruses, and are considered central components of the innate immune system⁶⁻⁸.

Although overall AMPs exhibit remarkable sequence and structural diversity, commonalities include a typical length less than 100 amino acids, a positive net charge, and membership in one of four distinct groups based on their secondary structures: β-strand, α-helix, extended coil, and loop. Of these groups, α-helix AMPs are the most studied and most common^{6, 9, 10}. The cationic nature of AMPs, along with a distribution of hydrophobic residues, enable these peptides to interact with and neutralize pathogens, and contribute to their overall function^{6,11}.

AMP structure may show variability across the tree of life¹². Amphibian AMPs are generally composed of an N-terminal signal peptide presequence, an adjacent prosequence that functions to maintain the AMP in an inactive conformation, and a C-terminal mature peptide sequence. All eukaryotic AMPs are synthesized as precursors that are proteolytically processed by propeptide convertases to yield active, mature peptides^{8,13-15}. While AMP signal peptides and prosequences are typically conserved within families, the mature peptide sequences vary considerably, and constitute the functional portion of the antimicrobial peptide⁸. These characteristics can be exploited to identify and characterize novel AMPs from a large dataset¹⁰. Furthermore, because of the multifaceted mechanisms of antimicrobial action employed by AMPs, such as destruction of microbial membranes¹⁶, inhibition of macromolecule synthesis¹⁷, and peptide-induced modulation of the immune system¹⁸, microbes are less likely to develop resistance against these peptides than against conventional antibiotics. Several AMPs are currently used in a clinical setting, and many more AMPs are undergoing clinical trials to ascertain their therapeutic potential¹¹.

The predominant approach for isolating new AMPs involves chromatography- and/or mass spectrometry-based analyses of protein samples from body fluids or tissues in combination with antimicrobial assays, peptide sequencing, and de novo peptide synthesis. However, context-specific protein expression, the cost of implementation, and low throughput experimentation associated with traditional AMP identification methods that employ analytical chemistry have hindered AMP discovery progress. This emphasizes the need to develop an alternative approach for the identification of novel AMPs with therapeutic potential.

Adult frog skin is an abundant source of AMPs due to specialized granular glands in the dermis that synthesize and store these peptides, which are secreted onto the skin surface at the first sign of injury or microbial challenge^{6,9,19}. From an evolutionary survival perspective, this rich repertoire of AMPs within frog skin is a beneficial adaptation to their wet and muddy environments where pathogens are plentiful. As of this writing, the curated Antimicrobial Peptide Database (APD)¹⁹ contains sequences for 978 active peptides originating from frog skin (out of 1043 amphibian peptides). This represents 34% of the AMP database compendium, which includes peptide sequences derived from six kingdoms including bacteria, archaea, protists, fungi, plants, and animals as well as some synthetic peptides (http://aps.unmc.edu/A P/main.php). Furthermore, the utility and efficacy of some frog AMPs as potential therapeutics has been demonstrated previously²⁰⁻²².

AMP secretion is not just limited to amphibians, nor limited to the skin or a specific developmental stage. For example, liver-expressed antimicrobial peptides (LEAPs) are highly abundant in the liver and midgut and, in humans and fish, are secreted into the blood²⁻²⁵³. As amphibians, most frogs experience life in two distinct postembryonic forms: as a free-living aquatic larval tadpole and as an air-breathing terrestrial frog. The demands on the innate immune system differ as the types of pathogens living in each environment can differ substantially. Therefore, there is an opportunity to identify novel AMPs expressed in the larval stage. Tadpole-specific studies conducted to date have focused on testing natural skin secretions collected from a mixture of different aged tadpoles after immersion in or injection of norephinephrine. This established that these skin secretions could defend against parasitic worm infection and survival²⁶. Using mass spectrometry, Woodhams and coworkers²⁷ compared the norepinephrine-induced skin secretions of 17 frog species and found that Ranatuerin-2, -4, -6, -7, -8, and -9, Palustrin-2CBa, Bradykinin, Temporin-1P, and Ranalexin were the most abundant peptides. Generally, tadpoles had a lower proportion of AMPs relative to adults, but their profiles are distinct from each other²⁷. Of these, Ranatuerin-2, -7, -8, -9, and Ranalexin were found even in the absence of norepinephrine induction²⁷. An interesting finding was that tadpoles with longer larval periods, such as that of R. catesbeiana, produced a greater AMP defense response than tadpoles with short larval periods showing differential investment in the innate immune response at this aquatic developmental phase²⁷.

Herein, the development of a bioinformatics approach for the identification and characterization of putative AMPs based on peptide homology is demonstrated. A manually curated AMP sequence database was used to search the rich genomic resources compiled for the North American bullfrog, Rana (Lithobates) catesbeiana²⁸. Two novel bullfrog AMPs were identified that demonstrate antimicrobial activity via an established microtiter broth dilution method²⁹. Through computational methods applied to transcriptomics and genome data, the expression profile and gene structures of twenty AMP-encoding transcripts were examined, sixteen of which are found in tadpole tissues.

### Results

Identification of putative AMP-encoding transcripts. A systematic stepwise in silico query of the Bullfrog Annotated Reference Transcriptome (BART²⁸) database is outlined in the Methods section and resulted in the identification of seven R. catesbeiana transcripts encoding novel precursor AMPs (shown in Table 1) and eleven known precursor AMPs (shown in Table 2).

Table 1 shows the characteristics of putative AMP sequences identified through bioinformatic analysis of bullfrog tadpole RNA-seq data. Each peptide sequence is separated into the prepro sequence and the presumed mature peptide sequence. Computational predictions of net charge, molecular weight (MW), and isoelectric point (pI) of the mature peptide are shown, as well as a Peptide ID for easy reference.

TABLE 1
Prepro Sequences and Putative Mature Peptide Sequences

| | Putative Mature Peptide | Net | | | Peptide |
|---|---|---|---|---|---|
| Prepro sequence | Sequence | Charge | MW | pl | ID |
| MFTMKKSLLLLFFLGTI | AFLSTVKNTLINVAGTMID | +2 | 3077.7 | 8.6 | HP2 |
| SLSLCEQERNADDDQGE | TFKCKITGVC | | | | |
| VIEQKVKR | (SEQ ID NO: 2) | | | | |
| (SEQ ID NO: 1) | | | | | |
| VLLYLIITVSFPRRDAN | SLSGCWTKSFPRKPCLRNR | +5 | 2236.6 | 10.9 | HP3 |
| DEDGGEVTKEVVKR | (SEQ ID NO: 4) | | | | |
| (SEQ ID NO: 3) | | | | | |
| MSSFCEITNVALTISLS | SMLSVLKNLGKVGLGFVAC | +4 | 2651.3 | 9.6 | HP4 |
| SPRRGADEEEGNGEKEI | KINKQC | | | | |
| KR | (SEQ ID NO: 6) | | | | |
| (SEQ ID NO: 5) | | | | | |
| MTQSTQKWFKTKYWRVR | NPSNLRALEELVKEECSEI | +1 | 3908.5 | 8.0 | HP5 |
| NRPAMSPDLNPIEHLWR | PVERCKKLIYGYRK | | | | |
| DLKKVVGKR | (SEQ ID NO: 8) | | | | |
| (SEQ ID NO: 7) | | | | | |
| MRKRMTMRRMMKKKKSE | MMRVMRRKTKVIWEKKDFI | +4 | 3144.8 | 10.2 | HP6 |
| KERRERGKR | GLYSID (SEQ ID | | | | |
| (SEQ ID NO: 9) | NO: 10) | | | | |
| MFFMSSPRRDADEVKEV | GFLDIIKNLGKTFAGHMLD | +2 | 3417.1 | 8.6 | HP8 |
| KR | KIKCTIGTCPPSP | | | | |
| (SEQ ID NO: 11) | (SEQ ID NO: 12) | | | | |
| MITVSSPRRDADGDEGE | GFLDIIKDIGKEFAVKILN | +2 | 3304.0 | 8.6 | HP9 |
| VEEVKR | NLKCKLAGGCPP | | | | |
| (SEQ ID NO: 13) | (SEQ ID NO: 14) | | | | |

The translated “novel” precursor AMPs include a trypsin cleavage site (a common convertase cleavage site in AMPs) and, apart from one sequence that begins with a valine (HP3), all have a methionine at the N-terminus (Table 1). Further, the putative mature peptides all possess a predicted net positive charge at neutral pH, are between 19 and 33 AA in length, and have isoelectric points (pI) between 8.0 and 10.9 (Table 1). Ag of these physicochemical properties are consistent with those of known AMPs¹⁹.

Table 2 shows transcript, protein, and gene characteristics of 20 known and putative AMPs evaluated within the present study.

TABLE 2
Transcript, protein, and gene characteristics of 20 known and
putative AMPs evaluated within the present study.

| | | | New Rana | |
|---|---|---|---|---|
| | Closest | Closest NCBI | catesbeiana | |
| | NCBI | precursor | transcripts | Rana catesbeiana gene information |
| | nucleotide | protein sequence | from BART | Genome scaffold/Strand/Scaffold |
| | sequence | Accession #/ | Accession #/ | length (bp)/[Range of overlap with |
| AMP | Accession #/ | Length (AA)/ | Length (nt)/ | query sequence by scaffold nt |
| name | Length (nt) | Species | Length (AA) | position]/Exon # |
| catesbeianin- | FJ830640/ | ACR84056/42/ | N/A^{a} | None |
| 1 | 324 | Rana/ | | |
| | | catesbeiana | | |
| catesbeianin- | FJ830640/ | ACR84056/42/ | GFBS01479282/ | None |
| 1 | 324 | Rana/ | 626/51 | |
| (HP6) | | catesbeiana | | |
| cathelicidin- | KF766531/ | AHW58221/ | MH800186/ | None |
| like-2 | 700 | 156/Rana/ | 753/155 | |
| | | catesbeiana | | |
| cathelicidin- | JF923766/ | AEI69698/179/ | MH800187/ | Rc-03r170621s387134/Rc- |
| AL | 648 | Amolops/ | 1019/181 | 03r170621s67282/+/−/1650/ |
| | | loloensis | | 14834/[1-400/9076-8969, 7977- |
| | | | | 7888, 5747-5490]/4 |
| cathelicidin- | KF766530/ | AHW58220/ | MH800188/ | None |
| RC1 | 677 | 151/Rana | 926/151 | |
| | | catesbeiana | | |
| cathelicidin- | KF766531/ | AHW58221/ | MH800189/ | None |
| RC2 | 700 | 156/Rana | 753/155 | |
| | | catesbeiana | | |
| HP5 | None | None | GFBS01753449/ | Rc-03r170621s32519/+/36533/ |
| | | | 1523/76 | [3251-4772]/1 |
| leap2 | XM_ | XP_01841872281/ | MH800190/ | Rc-03r170621s1377; Rc- |
| | 018563220/ | Nanorana | 3507/81 | 03r170621s5616/ |
| | 469 | parkeri | | +/+/[279751/290780]/[62065- |
| | | | | 62169, 73789-73937, 77722-79756/ |
| | | | | 141039-142257]/4 |
| palustrin- | FJ830669/ | ACR84085/ | N/A | Rc-03r170621s223822/Rc- |
| Ca | 322 | 71/Rana | | 03r170621s451975/+/+/[2611/ |
| | | catesbeiana | | 1423]/[2144-2217/128-343]/2 |
| palustrin- | FJ830669/ | ACR84085/ | GFBS01150567/ | Rc-03r170621s46320^{b}/+/24012/ |
| Ca | 322 | 71/Rana | 527/54 | [18718-19222]/1 |
| (HP9) | | catesbeiana | | |
| ranacyclin- | FJ830643/ | ACR84059/ | N/A | Rc-03r170621s43867/+/25684/ |
| Ca | 311 | 63/Rana | | [10079-10152, 12881-13068]/2 |
| | | catesbeiana | | |
| ranacyclin- | FJ830643/ | ACR84059/63/ | GFBS01071740/ | Rc-03r170621s29221/−/41060/ |
| Ca | 311 | Rana | 1143/ | [20482-19341]/1 |
| (HP3) | | catesbeiana | 50^{c} | |
| ranacyclin- | FJ830653/ | ACR84069/67/ | GFBS01607132/ | Rc-03r170621s43867/−/25684/ |
| Cc | 296 | Rana | 524/62 | [8419-8475, 10070-10152, 12881- |
| | | catesbeiana | | 13075]/3 |
| ranatuerin- | FJ842524/ | ACR46972/66/ | N/A | Rc-03r170621s168979/−/3446/ |
| 1 | 314 | Rana | | [2612-2539, 1247-1038]/2 |
| | | catesbeiana | | |
| ranatuerin- | KZ060483/ | PIO12229/61/ | N/A | Rc-03r170621s168979/−/3446/ |
| 1 | 3446 | Rana | | [1475-1107]/1 |
| (HP4) | | catesbeiana | | |
| ranatuerin- | JQ511836/ | AFR43665/71/ | GFBS01116610/ | Rc-03r170621s5461/−/149396/ |
| 2PRc | 253 | Pseudacris | 772/71 | [114249-114211/108748-108668/ |
| (HP2) | | regilla | | 100045-99394]/3 |
| ranatuerin- | FJ830657/ | ACR84073/74/ | GFBS01229406/ | Rc-03r170621s59711/Rc- |
| 2RC | 335 | Rana | 500/74 | 03r170621s128997/+/−/[17230/ |
| | | catesbeiana | | 5085]/[7179-7319, 11174-11256/ |
| | | | | 4558-4326]/3 |
| ranatuerin- | FJ830656/ | ACR84072/68 | N/A | Rc-03r170621s223822/Rc- |
| 3RC | 309 | Rana | | 03r170621s584290/+/−/2611/ |
| | | catesbeiana | | 1023]/[2144-2217/734-530]/2 |
| ranatuerin- | FJ830656/ | PIO09118/51/ | GFBS01228991/ | Rc-03r170621s584290/−/1023/ |
| 3RC | 309 | Rana | 519/51 | [1007-532]/1 |
| (HP8) | | catesbeiana | | |
| ranatuerin- | BT081520/ | ACO51651/70/ | GFBS01229403/ | Rc-03r170621s71023/Rc- |
| 4 | 332 | Rana | 504/70 | 03r170621s251277/+/−/[13560/ |
| | | catesbeiana | | 2355]/[8821-8968, 10977-11059/ |
| | | | | 2198-1964]/3 |

^{a}N/A indicates that the sequence found in the BART database was the same length as the Rana catesbeiana sequence already present in the NCBI database or that this sequence was not found in the BART database.
^{b}This scaffold contained sequence that was 93% identical to the HP9 sequence but had 17 AA changes and an in-frame deletion of V36
^{c}Translation begins with a V instead of M

Examination of putative AMP protein sequences. Blastx analyses of the seven transcripts identified protein sequence matches in the NCBI nr database ranging from 49-77% sequence identity (Table 3) and one sequence (HP5) had no notable match with any known AMP.

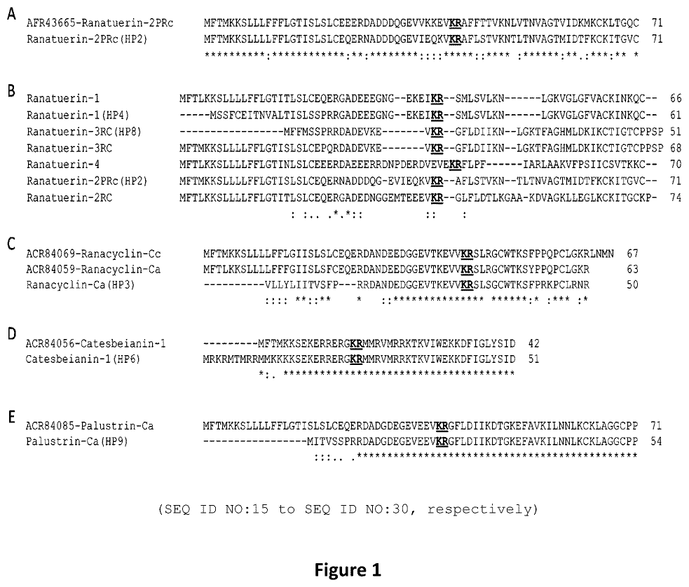

FIG. 1 shows Clustal omega alignments of putative AMP precursor sequences with their closest known AMP matches. Panel A shows a comparison of the Pseudacris regilla Ranatuerin-2PRc sequence (top) (SEQ ID NO:15) with the R. catesbeiana HP2 sequence (bottom) (SEQ ID NO:16). Panel B shows alignments of seven Ranatuerin precursor sequences (SEQ ID NO:17 to SEQ ID NO:23) from R. catesbeiana. Panel C shows alignments of Ranacyclin precursor sequences from R. catesbeiana (SEQ ID NO:24 to SEQ ID NO:26). Panel D shows alignments of Catesbeianin-1 precursor sequences (SEQ ID NO:27 and SEQ ID NO:28) from R. catesbeiana. Panel E shows alignments of Palustrin-Ca precursor sequences (SEQ ID NO:29 and SEQ ID NO:30) from R. catesbeiana. The conserved proteolytic cleavage site is shown in bold and underlined. This cleavage site indicates the border for the N-terminal prepro sequence and the C-terminal mature sequence. The precursor peptide lengths are indicated to the right of each sequence. The dots represent conserved amino acid substitutions and asterisks indicate exact matches. Dashes were introduced to maximize sequence alignments. Further details regarding NCBI accession numbers are in Table 2.

Table 3 shows a comparison of sequence identities (%) of the AMP candidates with their best-known AMP blastp matches over the entire sequence (precursor), or by prepro sequence or mature sequences. There was no AMP match with HP5.

TABLE 3
Peptide Sequence Identities

| | Highest scoring | Sequence Identity (%) | | |
|---|---|---|---|---|
| Peptide ID | blastp match | Precursor | Prepro | Mature |
| HP2 | Ranatuerin-2PRc | 77 | 83 | 69 |
| HP3 | Ranacyclin-Ca | 49 | 41 | 68 |
| HP4 | Ranatuerin-1 | 68 | 49 | 100 |
| HP5 | None | None | None | None |
| HP6 | Catesbeianin-1 | 76 | 54 | 100 |
| HP8 | Ranatuerin-3RC | 65 | 33 | 100 |
| HP9 | Palustrin-Ca | 65 | 38 | 100 |

Closer examination of the peptide sequences revealed that four of the predicted mature peptides (HP4, HP6, HP8, HP9) are identical to known AMPs (Table 3 and FIG. 1), while the corresponding prepro regions exhibit identities ranging from 65-76% (Table 3 and FIG. 1). The remaining two candidate AMP peptides (HP2 and HP3) exhibited 69% (HP2) and 68% (HP3) identity to their best-known AMP mature peptide sequence matches (Table 3). The HP2 and HP3 prepro sequences also show considerable divergence from their best-known AMP match (amino acid identities of 77% and 49%, respectively; Table 3).

The HP2 sequence is the R. catesbeiana counterpart of the Pseudacris regilla Ranatuerin-2PRc sequence (FIG. 1, Panel A). When compared to the other known or putative AMP precursors, the Ranatuerin-2PRc (HP2) sequence exhibits a reasonable degree of sequence conservation with other Ranatuerins in the prepro sequence, but considerable divergence in the mature peptide (FIG. 1, Panel B). The putative mature peptide sequences of HP4 and HP8 are identical to the mature peptides of Ranatuerin-1 and Ranatuerin-3RC, respectively, but each has a distinct prepro sequence (FIG. 1, Panel B). The mature peptide region of HP3 is 68% identical to Ranacyclin-Ca, with substantial sequence divergence in the prepro sequence (41% identity; FIG. 1, Panel C). The mature peptide regions of HP6 and HP9 are identical to Catesbeianain-1 and Palustrin-Ca, respectively, but have divergent N terminal ends in the prepro sequences (FIG. 1, Panel D and Panel E).

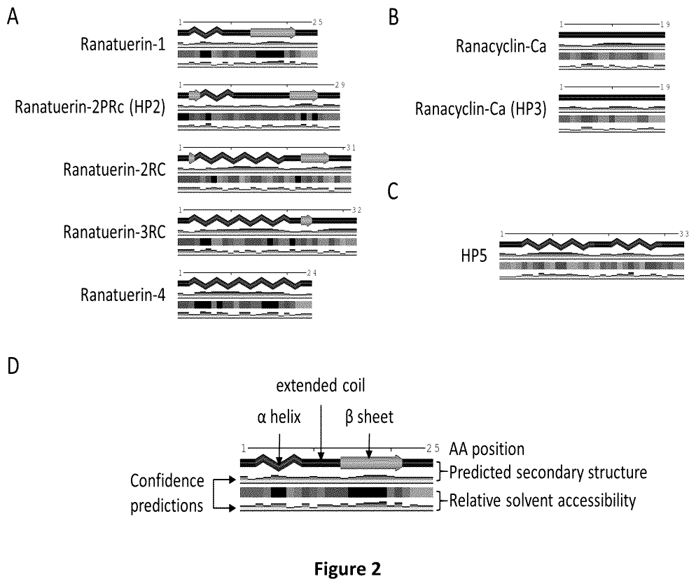

FIG. 2 shows SABLE secondary structure prediction comparisons between the derived mature peptides of Panel A—HP2; Panel B—HP3; and Panel C—HP5 versus known mature AMP sequences. Panel D shows the legend for the SABLE predictions with amino acid (AA) position indicated at the top, the predicted secondary structure in the middle and the relative solvent accessibility (RSA) at the bottom. Confidence predictions are below the predicted secondary structure and RSA. For the predicted secondary structure, red lines, a helices; green arrows, 3 sheets; blue lines, extended coils. RSA is indicated by grey scale from black (0-9% RSA) to white (90-100% RSA) where each box represents an amino acid.

The secondary structure of the putative mature HP2 peptide contains an α-helix, extended coil, β-strand arrangement that resembles a mixture of Ranatuerin-1 and Ranatuerin-2RC secondary structure FIG. 2, Panel A. The putative mature HP3 peptide is solely extended coil similar to Ranacyclin-Ca (FIG. 2, Panel B) while the putative mature HP5 peptide is comprised of two α-helices separated by a small extended coil (FIG. 2, Panel C).

Microtiter broth dilution assays. HP2, HP3, and HP5 peptides are comprised of novel sequences that have not yet been described in the AMP literature. A common method for establishing antimicrobial activity of peptides is to perform microtiter broth assays. Microtiter broth dilution methods were implemented for determination of the minimum inhibitory concentration (MIC) and minimum bactericidal concentration (MBC). HP3 and HP5 were tested in addition to Ranatuerin-1/HP4 peptide as a positive control. HP2 could not be tested because multiple peptide synthesis attempts failed. The human cathelicidin, LL-37³⁰ was used as an additional positive control, and an unrelated similarly sized cationic peptide from the T. pallidum protein Tp0751 was used as a negative control. The potential AMPs were tested against bacteria representative of all three types of known cell envelope (Gram-positive, negative, and the complex and unique mycobacterial cell wall/envelope), given that a major mechanism used by AMPs is cell wall/membrane targeting.

Five bacterial species were tested, spanning Gram-negatives (Escherichia coli and Pseudomonas aeruginosa), Gram-positives (Staphylococcus aureus and Streptococcus pyogenes), and Mycobacterium smegmatis (neither a true Gram-positive nor Gram-negative). The Ranatuerin-1 peptide had some activity against E. coli and S. aureus (MIC: 48 and 97 μM, respectively) which is higher than previously reported³¹ and some bactericidal activity was observed (MBC: 12-48 and 97 μM, respectively). This peptide had no effect on S. pyogenes or P. aeruginosa. HP3 and HP5 had no effect on inhibitory or bacteriostatic activity against E. coli, S. aureus, S. pyogenes, or P. aeruginosa.

Table 4 shows minimum inhibitory concentrations (MIC) and minimum bactericidal concentrations (MBC) in μM against M. smegmatis for tested known and putative AMPs from a minimum of five (MIC) and three (MBC) independent experiments. LL-37 is a human cathelicidin positive control and Tp0751 is a negative control peptide from T. pallidum. “-”, no effect observed.

TABLE 4
Minimum Inhibitory Concentrations and
Minimum Bactericidal Concentrations

| | μM | |
|---|---|---|
| Peptide Name | MIC | MBC |
| HP3 | 4-14 | 7-14 |
| HP4/Ranatuerin-1 | 2-12 | 3-48 |
| HP5 | 66 | ≥66 |
| LL-37 | 0.4-2 | 1-28 |
| Tp0751 | — | — |

Except for the negative control peptide, all peptides had bacteriostatic activity against M. smegmatis (Table 4). Compared to Ranatuerin-1, HP3 had comparable bacteriostatic activity (MIC: 4-14 versus 2-12 μM; Table 4), and better bactericidal activity (MBC: 7-14 versus 3-48 μM; Table 4). HP5 exhibited weak bacteriostatic and bacteriocidal activity against M. smegmatis (Table 4).

Expression of AMP-encoding transcripts in R. catesbeiana tadpole tissues. The abundance levels of twenty AMP-encoding transcripts (thirteen known and seven novel identified above; listed in Table 2) were assessed in premetamorphic R. catesbeiana tadpole back skin, liver, olfactory epithelium, and tail fin using normalized RNA-seq data from previous studies^{28,33}.

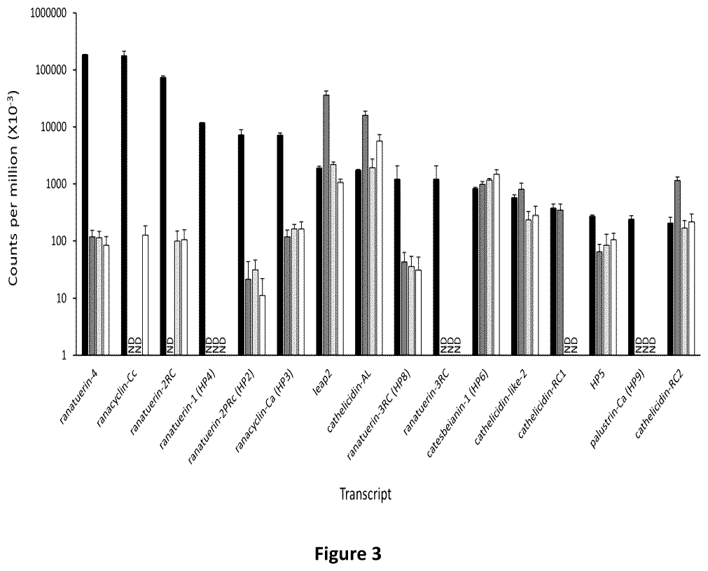

FIG. 3 shows putative and known AMP-encoding transcripts show differential expression in R. catesbeiana premetamorphic tadpole back skin, liver, olfactory epithelium, and tail fin. RNA-seq data representing transcripts encoding the indicated putative and known AMPs are shown for tadpole back skin (black bars, n=3), liver (dark grey bars, n=15), olfactory epithelium (light grey bars, n=15), and tail fin (white bars, n=15). Bars represent normalized median read counts per million and whiskers represent median absolute deviation. ND, not detected.

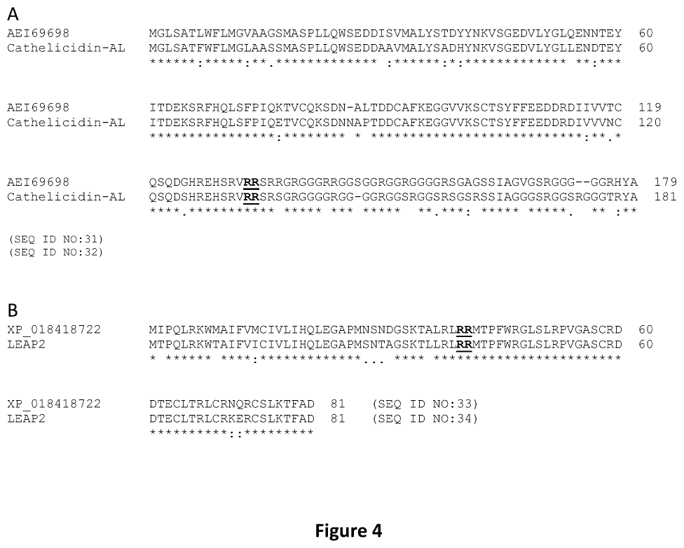

FIG. 4 shows Clustal omega alignments of putative AMP precursor sequences with their closest known AMP matches. Panel A shows a comparison of the Amolops loloensis Cathelicidin-AL sequence (NCBI Accession #AE169698; top) with the corresponding R. catesbeiana Cathelicidin-AL sequence (bottom). Panel B shows a comparison of the Nanorana parkeri predicted LEAP2 sequence (NCBI Accession #XP_018418722; top) with the corresponding R. catesbeiana LEAP2 sequence (bottom). The conserved proteolytic cleavage site is bold and underlined. This cleavage site indicates the border for the N-terminal prepro sequence and the C-terminal mature sequence. The precursor peptide lengths are indicated to the right of each sequence. The dots represent conserved amino acid substitutions and asterisks indicate exact matches. Dashes were introduced to maximize sequence alignments. Further details regarding NCBI accession numbers are in Table 2.

Sixteen of these transcripts are in one or more of these tissues (FIG. 3). A II of the indicated transcripts in FIG. 3 were sequence-verified from R. catesbeiana contigs including transcripts encoding Cathelicidin-AL (87% identity with Amolops loloensis precursor protein; FIG. 4) and LEAP2 (88% identity with Nanorana parkeri predicted precursor protein; FIG. 4). In several cases, the RNA-seq-derived sequences provided substantial improvements in length over transcript sequences already curated in GenBank (Table 2).

All sixteen transcripts are present in the tadpole back skin, while 11 are in the liver and olfactory epithelium, and 12 in the tail fin (FIG. 3). The most abundant transcripts are ranatuerin-4, ranacyclin-Cc, ranatuerin-2RC, and ranatuerin-1 (HP4) in the back skin; leap2, cathelicidin-AL, cathelicidin-RC2, and catesbeianin-1 (HP6) in the liver; leap2, cathelicidin-AL, catesbeianin-1 (HP6), and cathelicidin-like-2 in the olfactory epithelium; and cathelicidin-AL, catesbeianin-1 (HP6), leap2, and cathelicidin-like-2 in the tail fin (FIG. 3). Of note are the transcripts that are not in these premetamorphic tadpole tissues such as catesbeianin-1, ranacyclin-Ca, ranatuerin-1, and palustrin-Ca. These transcripts, which are detected in adult frog skin³¹, are replaced by catesbeianin-1 (HP6), ranacyclin-Ca (HP3), ranatuerin-1 (HP4), ranatuerin-3RC (HP8), and palustrin-Ca (HP9) (FIG. 3).

Previous work indicated that mRNA s encoding some AMPs increase from very low or undetectable levels in tadpoles to high levels in the frog as a consequence of thyroid hormone-dependent metamorphosis³⁴⁻³⁷. These determinations were done with either whole tadpole homogenates^{34,36} or skin^{35,37}.

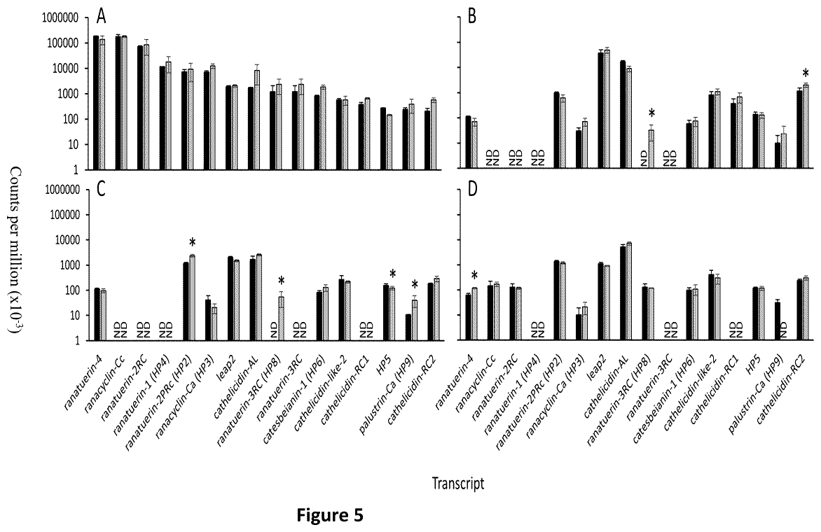

FIG. 5 shows Putative and known AMP-encoding transcripts generally are not responsive to 10 nM T₃ treatment of tadpoles. RNA-seq data representing transcripts encoding the indicated putative and known AMPs are shown for tadpole (Panel A) back skin (n=3), (Panel B) liver (n=5), (Panel C) olfactory epithelium (n=5), and (Panel D) tail fin (n=5) from vehicle controls (black bars) or tadpoles exposed to 10 nM T₃ for 48 h (grey bars). Bars represent median read counts and whiskers represent median absolute deviation. The asterisk indicates statistical significance between treatments at p<0.05. ND, not detected.

Premetamorphic tadpoles were immersed in 10 nM triiodothyronine (T₃) for 48 h which precociously induces metamorphosis by altering tissue-specific gene expression programs^{28, 33}, and determined the abundance of the AMP-encoding transcripts (FIG. 5). N one of these transcripts was responsive to T₃ in the back skin (FIG. 5, Panel A). While the vast majority of transcripts also were not responsive to T₃ treatment in the liver, olfactory epithelium, and tail fin (FIG. 5, Panel B to Panel D), ranatuerin-3RC (HP8) transcripts appeared in the liver and olfactory epithelium (FIG. 5, Panel B and Panel C). Significant increases in mRNA abundance were observed for cathelicidin-RC2 (2-fold) in the liver; ranatuerin-2PRc (HP2) (2-fold) and palustrin-Ca (HP9) (4-fold) in the olfactory epithelium; and ranatuerin-4 (2-fold) in the tail fin (FIG. 5, Panel B to Panel D). A slight but significant decrease (1.3-fold) in HP5 transcripts was observed in the olfactory epithelium (FIG. 5, Panel C). Palustrin-Ca (HP9) mRNA disappeared in the tail fin upon T₃ treatment, but this was not significant (FIG. 5, Panel D).

AMP-encoding gene structures in the bullfrog genome. It is possible that the new versions of the above transcripts were products of alternative gene splicing. U sing the recently published draft bullfrog genome²⁸, gmap and blastn were used to create gene models from the transcript sequences.

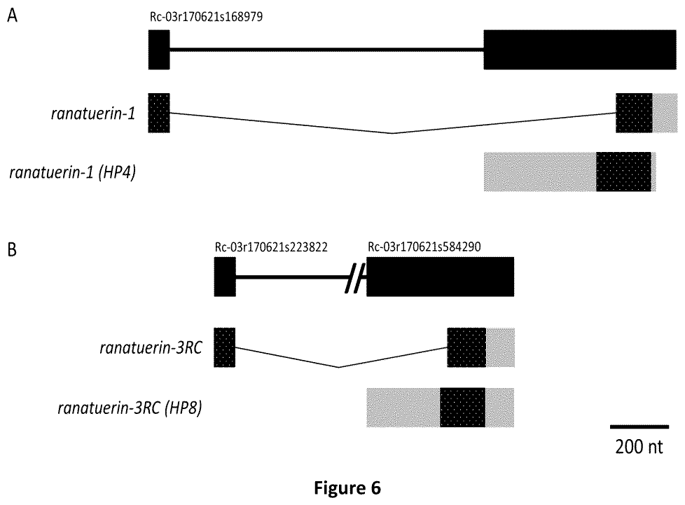

FIG. 6 shows the ranatuerin-1 and ranatuerin-3RC genes contain 2 exons and are alternatively spliced. The structure of the genes and derived transcripts encoding Panel A—Ranatuerin-1 and Ranatuerin-1 (HP4), and Panel B—Ranatuerin-3RC and Ranatuerin-3RC (HP8) are shown. The top illustration represents the corresponding gene drawn to the indicated scale with the exonic portions depicted as black rectangles and intronic regions depicted by the thick black line. The additional non-genic sequences flanking the indicated genes were present in all cases except where indicated. The NCBI v3.0 scaffold identifier from the bullfrog genome is indicated on the top left of each scaffold. Multiple scaffolds are indicated by a line break. Intronic regions are shown as thin lines that are spliced out in the labelled transcripts below the gene. The grey rectangles in the spliced transcript indicate the untranslated regions and the hatched rectangles indicate the open reading frame.

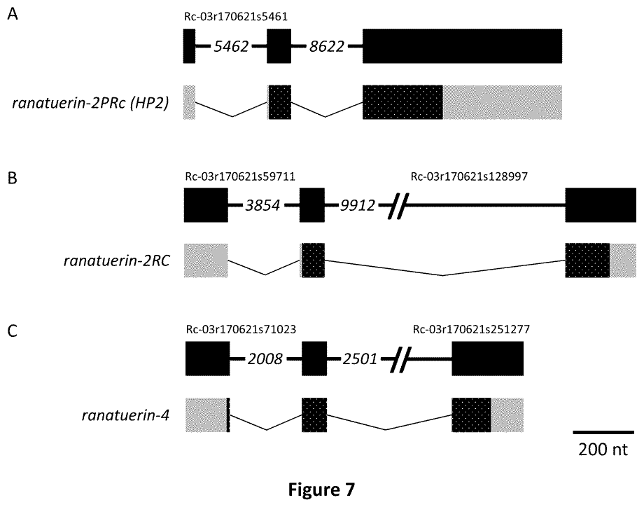

A 1.6 kbp two-exon gene gives rise to ranatuerin-1 and ranatuerin-1 (HP4) through alternative splicing (FIG. 6, Panel A) and a similar two-exon gene structure gives rise to ranatuerin-3RC and ranatuerin-3RC (HP8) through alternative splicing (FIG. 6, Panel B). In contrast, the three ranatuerins, ranatuerin-2PRc (HP2), ranatuerin-2RC, and ranatuerin-4, are each derived from distinct three-exon genes that are much larger (e.g. 15 kbp for ranatuerin-2PRc (HP2); see FIG. 7).

FIG. 7 shows the ranatuerin-2PR c (HP2), ranatuerin-2RC, and ranatuerin-4 genes are comprised of 3 exons. The structure of the genes and derived transcripts encoding Panel A—Ranatuerin-2PRc (HP2), Panel B—Ranatuerin-2RC, and Panel C—Ranatuerin-4 are shown. The illustrations are drawn to the indicated scale. The numbers in italics indicate the number of intervening base pairs where the intronic region was large. See FIG. 6 for more information.

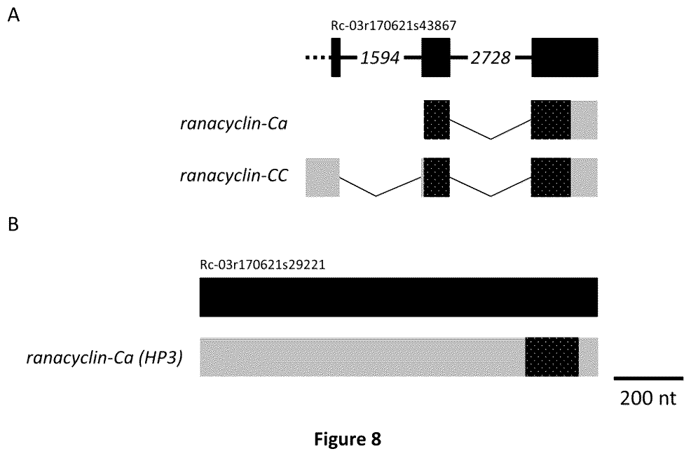

FIG. 8 shows two genes, one with 3 exons and the other with 1 exon, encode Ranacyclins. The structure of the genes and derived transcripts encoding Panel A—Ranacyclin-Ca and Ranacyclin-Cc, and Panel B—Ranacyclin-Ca (HP3) are shown. The illustrations are drawn to the indicated scale. The numbers in italics indicate the number of intervening base pairs where the intronic region was large. Refer to FIG. 6 for more information. The dotted line in “A” indicates that the 5′ end of the scaffold terminated prior to the available transcript information.

The transcripts encoding Ranacyclin-C a and Ranacyclin-CC come from the same 3-exon gene whereas the gene encoding ranacyclin-Ca (HP3) is comprised of a single exon on a different scaffold (FIG. 8). A similar relationship occurs for Palustrin-Ca. H ere, the palustrin-Ca transcript is derived from two exons (FIG. 8, Panel A) and palustrin-Ca (HP9) from a different single exon (FIG. 8, Panel B) from the same gene.

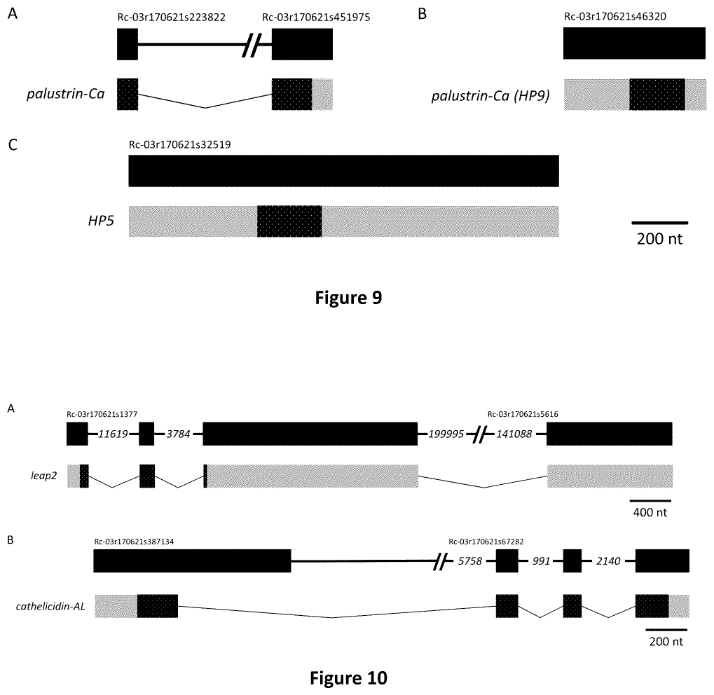

FIG. 9 shows Palustrin-Ca, encoded by a 2 exon gene, Palustrin-Ca (HP9) and HP5 are encoded by single exon genes. The structure of the genes and derived transcripts encoding Panel A—Palustrin-Ca; Panel B—Palustrin-Ca (HP9); and Panel C—HP5 are shown. The cartoons are illustrations to the indicated scale. The numbers in italics indicate the number of intervening base pairs where the intronic region was large. Refer to FIG. 6 for more information.

The gene encoding HP5 is comprised of a single exon (FIG. 9, Panel C). Finally, the leap2 and cathelicidin-AL transcripts are examples derived from the splicing of four exons (FIG. 10). The fact that all assembled transcript sequences above align with the independently-derived bullfrog genome with canonical splice sites further supports the legitimacy of the identified AMP transcript sequences.

FIG. 10 shows that both LEAP2 and Cathelicidin-AL are derived from four exons. The structure of the genes and derived transcripts encoding R. catesbeiana Panel A—LEAP2 and Panel B—Cathelicidin-AL, are shown. The illustrations are drawn to the indicated scale. The numbers in italics indicate the number of intervening base pairs where the intronic region was large. FIG. 6 provides additional information.

### Discussion

By utilizing known sequence homology and structural characteristics of AMPs from empirically validated peptides, a bioinformatics approach was applied to an assembled bullfrog tadpole reference transcriptome, and identified transcripts encoding putative novel AMPs and augmented the sequence information available for known AMPs. Historically, frogs are a rich source of AMPs. However, studies on larval tadpole stages have been limited, particularly those pertaining to gene expression studies.

The present study focused on the premetamorphic tadpole as an organism that is primarily dependent upon the innate immune system for microbial protection. Similar to what was observed in the frog, tadpole tissues express several AMPs with the greatest concentration in the back skin. Previous work in Xenopus laevis indicated that transcripts encoding magainin and “peptide with amino terminal glycine and carboxy terminal leucinamide” (PGLa) are not detected until metamorphic climax and into the frog stage³⁴. The abundance of these mRNAs increase in whole premetamorphic tadpoles by prolonged immersion in 5 nMT₃ for 7 d, inducing precocious metamorphosis³⁴. Other studies established that mRNAs encoding Ranalexin in R. catesbeiana³⁵, Brevinin-1SY in R. sylvatica³⁶ and Preprotemporin in R. ornativentris³⁷ generally transition from undetectable or very low levels in the tadpole through thyroid hormone-dependent metamorphosis to high levels in the frog. An induction of Preprotemporin-encoding mRNA upon injection of adult R. ornativentris with 2×10⁻⁹ M T₃ was observed³⁷. The abundance levels of twenty known and putative AMP-encoding transcripts were examined, of which sixteen were expressed in at least one of the four premetamorphic tadpole tissues in the present study. The vast majority of AMP-encoding transcripts were not affected by T₃ treatment after 48 h and none was hormone-responsive in the back skin. It is difficult to compare the previous studies with the current results for multiple reasons: the use of whole tadpole homogenates rather than specific tissues and/or the use of adults instead of tadpoles for T₃ injection studies. It is possible that longer T₃ exposure times may result in modulation of more AMP-encoding transcripts, but this remains to be determined. The data suggest that the metamorphosis-dependent change in AMP expression may be a later indirect thyroid hormone-dependent response leading to a resetting of the innate immune system coinciding with life transition.

The antimicrobial properties of Catesbeianin-1, Ranacyclin-Ca, Ranatuerin-1, Ranatuerin-3RC, and Palustrin-Ca have been known for some time⁶⁻⁸. This example represents the discovery that there can be diversity in their prepro sequences while retaining the mature peptide sequence of the respective AMP as a consequence of alternative splicing. An intriguing possibility is that the gene splice variants may be developmentally regulated as part of resetting the immune system during postembryonic development. The consequence of this shift embodies a change in prepro sequence rather than the mature peptide of the respective AMP. This may have regulatory consequences for peptide localization, processing and/or activation that have yet to be determined, and may possibly reflect a developmental shift in expressed activating proteases as well³⁸. Further examination into the expression profiles of the splice variants during development and in different tissues is warranted.

While considerable efforts have been placed on phylogenetic comparisons of AMPs at the protein level (for e.g. ^{39,40}), much less is understood regarding the structure of the genes giving rise to AMP-encoding transcripts. The current study presents the first gene structure information of AMPs with known antimicrobial functionality in frogs, and it was found that a range of AMP gene structures were represented. The four-exon gene structure observed in R. catesbeiana cathelicidin is conserved with the human LL37 cathelicidin gene on chromosome 3 (NCBI Accession NM_004345.4) while the R. catesbeiana leap2 gene has four exons compared to three in fish and humans⁴¹. This apparent discrepancy may be due to the fourth exon comprised entirely of untranslated region (FIG. 10). As the R. catesbeiana leap2 gene structure is currently composed of two scaffolds, the possibility cannot be definitively discounted that the leap2 transcript may be an assembly artifact, but routine improvements to the bullfrog genome assembly will resolve this remote possibility.

The Ranatuerin-encoding genes are subject to alternative splicing and possess two or three exons in R. catesbeiana, and the close relationship between Ranatuerins and Ranacyclins are reflected in the retention of the three-exon gene structure. Further, the diversity of AMP mature peptide sequences have been suggested to be a consequence of gene duplications from an ancestral gene⁴². The present study provides support for this in addition to alternative splicing as another mechanism for AMP diversity.

Two new mature AMP sequence candidates, in addition to Ranatuerin-1, demonstrated antimicrobial activity against M. smegmatis. Of particular note, HP3 and the established AMP, Ranatuerin-1, exhibited similar antimicrobial activity against the mycobacterium, M. smegmatis. This species was used to establish that the novel AMPs described herein are active against Mycobacteria. Since all Mycobacterium species have a similar cellular structure, demonstrating activity against a classic non-pathogenic species has provided us the evidence that it is worthwhile to next assess the activity of the novel AMPs against pathogenic species in future experiments.

While there is some variability within the activity results presented herein, this Example clearly illustrate the process of designing optimized AMPs that exhibit improved consistency, reproducibility, stability, and enhanced activity⁴³. Sequence analysis of the new AMP candidates revealed diversity within the prepro and the mature peptide sequences adding to the growing assortment of AMPs. The linkage of known AMP sequences to new prepro sequences opens up new possibilities for further AMP candidate discovery. Successful functional testing of AMPs identified via the bioinformatics methods used in the present study affirms the value of using a bioinformatics approach to mine the bullfrog genome, as described herein.

Because AMPs play a critical role in innate immunity^{6,44}, further examination of the circumstances of their expression and factors that may disrupt their normal function could inform conservation efforts. Amphibians are experiencing drastically decreased population numbers worldwide due to infectious pathogens^{44,45}. The interplay between AMPs and pathogens is an important determinant of host survival upon infection, and some amphibian AMPs are known, for example, to kill the chytrid fungus, Batrachochytrium dendrobatidis^{46,47}. Resistance can be conferred by fungal secretion of a protease that cleaves and disrupts amphibian AMP function³³ revealing the need for further investigations into the mechanisms of AMP regulation and their relationship to disease protection and pathogen evasion. In addition, continued investigations into the wealth of natural antibiotic compounds produced by amphibians will also undoubtedly result in further discovery of novel AMPs that may lead to the development of effective therapeutics for combatting the major and increasing global health threat of antibiotic resistance.

### Sequence Availability

All biological sequences referenced herein by accession numbers, such as are available through NCBI, are hereby incorporated by reference as though the sequence was recited in its entirety within the subject text, figure, or tables.

### Methods

Further details of the methods used herein are provided below. Citations are provided to indicate further details, and all references pertaining to methodologies used herein are hereby incorporated by reference.

In silico prediction and characterization of putative antimicrobial peptides. Seven novel AMP candidates were initially identified from the bullfrog annotated reference transcriptome (BART version 3, NCBI TSA accession GFBS01000000)^{28,33} using the following three steps.

First, the BART transcript sequences, all of which were de novo assembled with Trans-ABySS⁴⁸ from strand-specific RNA-Seq libraries²⁸, were in silico translated using Transdecoder (-m 20 -S; version 2.0.1) (github.com) and complete predicted open reading frames up to 100 amino acids long were retained.

Second, Hidden Markov models (HMMs) representing the salient features of AMPs from 35 protein families were downloaded from the Collection of Antimicrobial Peptides database (bicnirrh.res.in), and HMMER⁴⁹ was used to identify BART peptide sequences with similarity to one or more HMM (default settings, significance considered at E<0.001). These hits were then further refined using InterProScan⁵⁰ default settings with the Pfam database⁵¹ of protein domain HMMs (version 29.0).

Third, candidate AMPs had to satisfy the following criteria: 1) the putative open reading frame began with a methionine or valine residue confirmed by Virtual Ribosome 2.0⁵² analysis, and 2) the protein sequence contained a canonical propeptide convertase Lys-Arg (KR) cleavage site as determined by ExPA Sy Peptide C utter (expasy.org). With the exception of one AMP candidate, all peptide sequences also had strong alignment to a known precursor AMP defined as an E-value score of <10⁻⁴ using blastx or blastp against the NCBI nr database. If the candidate AMP sequence had a full precursor alignment to a sequence in the NCBI nr database with identity and positivity scores of greater than 90%, then the sequence was considered “known”. A final set of seven “novel” and eleven “known” AMP-encoding R. catesbeiana transcripts were found from tadpole tissues (Table 2). An additional two AMP sequences that were already present in the NCBI nr database from previous studies on adult frogs were also examined in the present study (Table 2). Final protein alignments were generated using Clustal Omega version 1.4.2 (ebi.ac.uk)⁵³.

Secondary structures of the mature AMP peptides were assessed using SA B L E Protein prediction (cchmc.org). The net charge, molecular weight, and isoelectric points (pI) of the mature peptides were determined using ExPASy ProtParam (expasy.org).

Gene expression analysis. The levels of twenty AMP-encoding R. catesbeiana transcripts (Table 2) were determined in premetamorphic R. catesbeiana tadpole back skin, tail fin, olfactory epithelium, and liver tissues through RNA-seq data derived from previous studies of tadpole tissues^{28, 33}. Strand-specific mRNA libraries were constructed and sequenced via Illumina HiSeq and aligned to the BART reference transcriptome²⁸ to generate counts. All RNA-seq experiments had comparable sequencing depth and were normalized to the total number of reads per sample. To normalize the counts, the number of reads were divided by the total number of reads in the corresponding sample and multiplied by 100 million.

Gene structure determination. The longest cDNA sequence from each of twenty R. catesbeiana transcripts encoding AMPs (Table 2) was used to query the high quality draft bullfrog genome (NCBI Accession number LIA G00000000, BioProject PRJNA 285814)²⁸ using gmap version 2017-04-1354. The relevant scaffolds are indicated in Table 2.

Microtiter broth dilution assays. To test for antimicrobial activity, HP3, HP4/Ranatuerin-1, and HP5, peptides were synthesized by GenScript (Piscataway, New Jersey, USA). HP2 was not tested because the service provider was unable to synthesize this peptide despite multiple attempts. An unrelated, similarly-sized peptide from the Treponema pallidum protein Tp0751⁵⁵ was used as a negative control and the human cathelicidin, LL-37³⁰, was included as a positive control. Peptides were dissolved in filter-sterilized ultrapure water and tested for sterility by plating on non-selective agar plates followed by a 48 h incubation at 37° C. Two-fold serial dilutions of each peptide were prepared to obtain a series corresponding to ten times the required testing concentrations (2560, 1280, 640, 320, 160, 80, 40, 20, 10, and 5 μg/mL)²⁹.

Microtiter broth dilution methods were implemented for determination of the minimum inhibitory concentration (MIC) and minimum bactericidal concentration (MBC) of the four putative AMPs and the negative control peptide using procedures adapted from the R. E. W. Hancock Laboratory for cationic AMPs²⁹ and the CLSI methods for dilution antimicrobial susceptibility tests⁵⁶.

To assess antimicrobial activity across a diverse range of bacterial species, colonies were cultured overnight on Mueller Hinton agar plates (MHA; +5% sheep blood for S. pyogenes)⁵⁶ from frozen glycerol stock. Bacteria tested include Gram-negative rods (Escherichia coli: ATCC 9723H; Pseudomonas aeruginosa: ATCC 10148), Gram-positive cocci (Staphylococcus aureus: ATCC 6538P; Streptococcus pyogenes: unknown strain, hospital isolate), and Mycobacterium smegmatis (MC²155; classified as neither Gram-positive nor Gram-negative). Bacterial suspensions were prepared by placing 3-5 morphologically similar colonies from the grown plate into sterile glass culture tubes containing 2 mL of Mueller Hinton Broth (MHB; +5% lysed horse blood for S. pyogenes)⁵⁶. Microbial inoculums from bacterial suspensions were prepared through a spectrophotometric adjustment of turbidity to 0.08-0.1 at 600 nm to achieve a turbidity equivalent to that of a 0.5 McFarland standard (1-2×10⁸ CFU/mL)²⁹. The standardized bacterial inoculums were then diluted in MHB to obtain final cell densities of approximately 5.0×10⁵ CFU/mL.

Ninety-six-well microtiter plates (Fisher Cat. No. CS003790; Nepean, Ontario, Canada) were prepared with 100 μL of E. coli, P. aeruginosa, S. aureus, S. pyogenes, or M. smegmatis bacterial suspension (5×10⁵ CFU/mL) dispensed into each well of columns 1 through 11. Eleven microliters of the 10× AMP dilution series for all four peptides were added to each well from column 1 (2560 μg/mL) to column 10 (5 μg/mL) in all plates. Column 11 functioned as a positive control for bacterial growth in the absence of AMPs. Column 12 in each plate contained 100 μL of MHB as a sterility control (+5% lysed horse blood for S. pyogenes)⁵⁶. Plates were incubated at 37° C. for 16-24 within 15 min of adding the inoculum.

MIC values were visually determined by comparing the amount of bacterial growth (turbidity) in wells containing AMPs with growth in the control wells that did not contain any amount of peptide. MBC values were determined by plating the entire contents of the wells containing the peptide/bacteria mixture representing the MIC and the entire contents of the two preceding wells containing 2-fold and 4-fold more concentrated AMP dilutions/bacteria mixtures onto non-selective MHA plates, followed by incubation for 24 h at 37° C.

### Example 2

### Antimicrobial Peptides

The following peptide sequences, located as described herein, may be used for antimicrobial applications as described. The peptides were prepared and tested as outlined below.

Group P1
(SEQ ID NO: 66)
XXXPXXXXXGGK
(SEQ ID NO: 67)
FYFPXXXXXGGK
(SEQ ID NO: 68)
XYFXXSRKXXXX
(SEQ ID NO: 69)
FXFXVSRKXXXX
(SEQ ID NO: 70)
XYFXXXXKFXXK
(SEQ ID NO: 71)
XXXXXXXXFGGK
(SEQ ID NO: 72)
FYXPVXRXFXXX
Example sequence P1_CCH, ‘natural’
(SEQ ID NO: 35)
FYFPVSRKFGGK
Example sequence P1_CCH_F9R_Y2P, ‘synthetic’
(SEQ ID NO: 36)
FPFPVSRKRGGK
Group P2
(SEQ ID NO: 73)
FFPRXXXXXXXFLPTXXXXXXXSVGN
(SEQ ID NO: 74)
XXXXVLPLANKXXXXIYCXXXXXXXX
(SEQ ID NO: 75)
FFPXXLPLANXXLPXXXXXLPXXVGN
(SEQ ID NO: 76)
XXPRVLXXXXXXXXXXXXXXPKSXXX
(SEQ ID NO: 77)
FXXXXXXLANKXXXTIYCXXXXSVXX
(SEQ ID NO: 78)
FFXXVLPXXXXXLXTXYCALPKXVXN
(SEQ ID NO: 79)
XXXXXXXLANXFXPXIXXALPKXXGX
Example sequence P2_CCH, ‘natural’
(SEQ ID NO: 37)
FFPRVLPLANKFLPTIYCALPKSVGN
Example sequence P2_CCH_T15K_P7R, ‘synthetic’
(SEQ ID NO: 38)
FFPRVLRLANKFLPKIYCALPKSVGN
Group P3
(SEQ ID NO: 80)
GLLXXXXXXXXXXXXXXXXXXXXXXXXCPPSS
(SEQ ID NO: 81)
XXXXXXKXXXKXXGXLMXXXXXXMXGXXPPXS
(SEQ ID NO: 82)
GLLXIIXXXGXTTGILMXXLXXXMXGXXPPXX
(SEQ ID NO: 83)
XLXXXXXXXXKXXXXXXXTLKCQMTXXCXXSS
(SEQ ID NO: 84)
GLLXIIKXTGKTTGILMXXLKXXXXGXXXXXX
(SEQ ID NO: 85)
GXLXIIKXTGXXTXIXMXTLKCQXTGRXPPSS
(SEQ ID NO: 86)
GLLXXXKXTGKXTXIXXXTLKXQXTGRXXXXX
(SEQ ID NO: 87)
XXXXIIXXTXXTXGXLXXTXXCQXTXRXXXXX
Example sequence P3_CCH, ‘natural’
(SEQ ID NO: 39)
GLLDIIKDTGKTTGILMDTLKCQMTGRCPPSS
Example sequence P3_CCH_D8K_Q230, ‘synthetic’
(SEQ ID NO: 40)
GLLDIIKKTGKTTGILMDTLKCCMTGRCPPSS
Group P4
(SEQ ID NO: 88)
GLLXIIXXXGXXXXXXILXXLXXXLAGGXXX
(SEQ ID NO: 89)
GLLXXXKTTGKXFAVKILXNLXXXXXXXXPP
(SEQ ID NO: 90)
XXXXIIKTXXXXFAVXXXXXXKCKLAGGXXX
(SEQ ID NO: 91)
XXXXIIXXXXKXXXXKXXXNLKCKXXXXCPP
(SEQ ID NO: 92)
GLLXXXKTTGXXXXXXXLXNLKCXXAXXCXX
(SEQ ID NO: 93)
GLLXXXKXXXXXFAVXXLXXLXXXXXXXXPP
Example sequence P4_CCH, ‘natural’
(SEQ ID NO: 41)
GLLDIIKTTGKDFAVKILDNLKCKLAGGCPP
Example sequence P4_CCH_P31K, ‘synthetic’
(SEQ ID NO: 42)
GLLDIIKTTGKDFAVKILDNLKCKLAGGCPK
Group P5
(SEQ ID NO: 94)
XXXXXXXLAAKXXXSLVXXXXKKC
(SEQ ID NO: 95)
XFPIIAXLAAXVIPXLVXAVTXXX
(SEQ ID NO: 96)
FFPXXAXXXXKXXPXXXXXXXXXX
(SEQ ID NO: 97)
XXXXXXXLAAXVIPXLXXXXTXXX
(SEQ ID NO: 98)
FFPIIAXXXXXXXXXXVCAVTKKC
(SEQ ID NO: 99)
XXXIIXRXXXKVIXSXVCXVTKKC
(SEQ ID NO: 100)
FFPIIARLAAXVIXSLXCAVXXXX
Example sequence P5_CCH, ‘natural’
(SEQ ID NO: 43)
FFPIIARLAAKVIPSLVCAVTKKC
Example sequence P5_CCH_A19K, ‘synthetic’
(SEQ ID NO: 44)
FFPIIARLAAKVIPSLVCKVTKKC
Group P6
(SEQ ID NO: 101)
GLWETIKXXXKXXXXXXXXKXXXXXXGGCPP
(SEQ ID NO: 102)
XXXXXXXTTGXXXXXXXXXXXKCKXXXXCXX
(SEQ ID NO: 103)
XXXXTIXXXGXXIALXLLXXIXXXIAXXXPP
(SEQ ID NO: 104)
GLWETXKTTXXSXXLNLLDKIXXKIAXXXPP
(SEQ ID NO: 105)
XXXXXIKXXGKSIALXXXXKXKXKXXGGXXX
(SEQ ID NO: 106)
XXXXXXXXXXKSIAXNLLXXIXCXIAGGXXX
Example sequence P6_CCH, ‘natural’
(SEQ ID NO: 45)
GLWETIKTTGKSIALNLLDKIKCKIAGGCPP
Example sequence P6_CCH_S12K, ‘synthetic’
(SEQ ID NO: 46)
GLWETIKTTGKKIALNLLDKIKCKIAGGCPP
Group P7
(SEQ ID NO: 107)
ATAWXIXXXGMXXIIXIXIXXLXGXX
(SEQ ID NO: 108)
XXXWXIXXXGMQXXXXXXXXXXCGKQ
(SEQ ID NO: 109)
XXXXXXPPPXXQPXXPXXXXPXXXXX
(SEQ ID NO: 110)
ATAXXXPPPXXXPXXPXXXXPXCXKQ
(SEQ ID NO: 111)
XXXWXIXXXGMXXXXXIXIXXLXGXX
(SEQ ID NO: 112)
XXXXXXXXXXXXPXXPXXIXXLXGXX
(SEQ ID NO: 113)
ATAXRXXXXXXQXIIXIRIRXLCXKQ
Example sequence P7_CCH, ‘natural’
(SEQ ID NO: 47)
ATAWRIPPPGMQPIIPIRIRPLCGKQ
Example sequence P7_CCH_P9R_R5M, ‘synthetic’
(SEQ ID NO: 48)
ATAWMIPPRGMQPIIPIRIRPLCGKQ
Group P8
(SEQ ID NO: 114)
FPAIIXXXXXXX
(SEQ ID NO: 115)
FXXXXCXXXKXC
(SEQ ID NO: 116)
XXAIIXXVSKXX
(SEQ ID NO: 117)
XPAXXCKXXXXX
(SEQ ID NO: 118)
FPXXXXXVSKNC
(SEQ ID NO: 119)
XXXIICKVSXNX
Example sequence P8_CCH, ‘natural’
(SEQ ID NO: 49)
FPAIICKVSKNC
Example sequence P8_CCH_N11K, ‘synthetic’
(SEQ ID NO: 50)
FPAIICKVSKKC
Group P9
(SEQ ID NO: 120)
FLTFXGXXFGXXXGX
(SEQ ID NO: 121)
XXXXPGMXFXXLLXX
(SEQ ID NO: 122)
XXXXPGMXXXKXXXK
(SEQ ID NO: 123)
FLTFXXXXXXXLLGX
(SEQ ID NO: 124)
FXXFXXXTFGKXXXK
(SEQ ID NO: 125)
XLTXXXXTFGKLLGK
Example sequence P9_CCH, ‘natural’
N/A
Example sequence P9_CCH_2, ‘synthetic’
(SEQ ID NO: 51)
FLTKPGMTFGKLLGK
Group P10
(SEQ ID NO: 126)
XXXXFFXVNIFXLXX
(SEQ ID NO: 127)
SNXXXXXVXXXRXXX
(SEQ ID NO: 128)
XXXXFFXXXIFXLCG
(SEQ ID NO: 129)
SNXXXXKXXXXXLCG
(SEQ ID NO: 130)
XXRXXXKVNIFXXCX
(SEQ ID NO: 131)
SXXXFFXVXIXXXXG
(SEQ ID NO: 132)
SXRDFFKXNXXRXCX
Example sequence P10_CCH, ‘natural’
(SEQ ID NO: 52)
SNRDFFKVNIFRLCG
Example sequence P10_CCH_2, ‘synthetic’
(SEQ ID NO: 53)
SNRKFFKVRIFRLCG
Group P11
(SEQ ID NO: 133)
XXXXXIQKXXXXNTLKXXKXXLXXX
(SEQ ID NO: 134)
ALVAKXXXFPVFXXXXLCXLXXXXX
(SEQ ID NO: 135)
ALVAKIQKXXXXXXXXXXKLXXXII
(SEQ ID NO: 136)
XXXXXXXKXPXXNTLKXCKXEXEXX
(SEQ ID NO: 137)
XXXXXXQXFXVFXTLKLXKLXLXXX
(SEQ ID NO: 138)
XLVAKIXXXPVXNXXXLXXXXLXII
(SEQ ID NO: 139)
AXXAXIXXFXXFXXXXXCXXEXEII
Example sequence P11_CCH, ‘natural’
(SEQ ID NO: 54)
ALVAKIQKFPVFNTLKLCKLELEII
Example sequence P11_CCH_E21K_E23R, ‘synthetic’
(SEQ ID NO: 55)
ALVAKIQKFPVFNTLKLCKLKLRII
Group P12
(SEQ ID NO: 140)
XXGQVXXXKXKXX
(SEQ ID NO: 141)
IAXXXAAAXXXXX
(SEQ ID NO: 142)
XXGXXXXXKXKHI
(SEQ ID NO: 143)
IXXQVXXXKQKHI
(SEQ ID NO: 144)
XXXQVXXXXQXHI
Example sequence P12_CCH, ‘natural’
(SEQ ID NO: 56)
IAGQVAAAKQKHI
Example sequence P12_CCH2, ‘synthetic’
(SEQ ID NO: 57)
IAGQKARAKQKHI
Group P13
(SEQ ID NO: 145)
XXRXPXXXXXKLWKXXLXXX
(SEQ ID NO: 146)
IQXXXVXXXLXXXXLXXXII
(SEQ ID NO: 147)
XXXLXXXNMXXXWKXXXXXX
(SEQ ID NO: 148)
XXXLPXINMXKLXXXXLXXX
Example sequence P13_CCH, ‘natural’
(SEQ ID NO: 58)
IQRLPVINMLKLWKLELEII
Example sequence P13_CCH_N8K_E18K, ‘synthetic’
(SEQ ID NO: 59)
IQRLPVIKMLKLWKLELKII
Group P14
(SEQ ID NO: 149)
IQRLXXXXXXXSLYXXXCRTC
(SEQ ID NO: 150)
XXXLPVXVXLPSLYXXXXXXX
(SEQ ID NO: 151)
IQRXXXIVIXXXXXCVIXXXX
(SEQ ID NO: 152)
XXXXPVXXXXPSLYXXXCRTC
(SEQ ID NO: 153)
IQRLXVIXILXXXXCXXCXXC
(SEQ ID NO: 154)
XXXLPXXXXXPXXXXVIXXTX
(SEQ ID NO: 155)
XXXLXVIVILXSLYCVICRTC
Example sequence P14_CCH, ‘natural’
(SEQ ID NO: 60)
IQRLPVIVILPSLYCVICRTC
Example sequence P14_CCH_2, ‘synthetic’
(SEQ ID NO: 61)
IQRLPVIVILPSLYCVICRKK
Group
(SEQ ID NO: 156)
LXXPXPXYXFXXGIGXXXXWXXXWLNAQQMXXXXX
(SEQ ID NO: 157)
XXCPTPXXXFXXXXXNHLXXXIIWLXXXXMXXXXX
(SEQ ID NO: 158)
LRCXXXXXXXENGXXXXXMWNXXXXXXXXXSYKNK
(SEQ ID NO: 159)
XXXXTXHYNXENGIGNHLMXNXXXXNXQXXXXKXK
(SEQ ID NO: 160)
LXCXXXHXNXXXXXGNHXXWXXXWLXXXXMSXXNX
(SEQ ID NO: 161)
XRXPXPHXXFXXXXXXXXXXXIIXXXAXQXSYXXX
Example sequence P15_CCH, ‘natural’
(SEQ ID NO: 62)
LRCPTPHYNFENGIGNHLMWNIIWLNAQQMSYKNK
Example sequence P15_CCH_2, ‘synthetic’
(SEQ ID NO: 63)
LRCPTPHYRFENGIGNHLMWNIIWLNAQQMSYCNK
Group P16
(SEQ ID NO: 162)
SNRXXXMXXXXGLXGPXXIMXXXXX
(SEQ ID NO: 163)
XXXXFFMXXXXXXCXXFGXXXXKXX
(SEQ ID NO: 164)
SNRDXXXXXIFGLXGPXXIMXRKRR
(SEQ ID NO: 165)
SXXXXXXVNXXXXCXXFGXXEXXXX
(SEQ ID NO: 166)
XXXXXXXVNIFXXXXPXXXXXRXRR
Example sequence P16_CCH, ‘natural’
(SEQ ID NO: 64)
SNRDFFMVNIFGLCGPFGIMERKRR
Example sequence P16_CCH_2, ‘synthetic’
(SEQ ID NO: 65)
SNRKFFMVNIFGLCGPFGIMKRKRR

### Methods

Peptides were tested for activity using as minimum inhibitory concentration (MIC), and minimum bactericidal concentration (MBC) determinations. For certain peptides, a hemolysis test was also conducted to determine HC₅₀ (μg/mL) as an indicator of hemolytic activity based on the concentration of an antimicrobial compound that kills 50% red blood cells.

Peptides were tested for activity against E. coli, S. aureus, P. aeruginosa, S. pyogenes, and M. smegmatis.

Independent experiments were conducted with four strains of E. coli, including ATCC E. coli (Escherichia coli—ATCC 25922 “wild type”); ESBL E. coli (Escherichia coli—Extended spectrum beta-lactamase); CPO E. coli (KPC) (Escherichia coli—Carbapenemase-producing organism; Klebsiella pneumoniaecarbapenemase); and CPO E. coli (NDM) (Escherichia coli—Carbapenemase-producing organism; New-Dehli Metallobetalactamase).

Independent experiments were conducted with different strains of Staphylococcus aureus—ATCC 29213 “wild type”; and Staphylococcus aureus—Methicillin resistant Staphylococcus aureus.

The ATCC (American Type Culture Collection) strains received from Cedarlane. Multi drug resistant (MDR) strains, as clinical isolates, were received from the laboratory of Dr. Linda Hoang.

Putative antimicrobial peptides were synthesized by GeneScript. A-list antimicrobial peptides re-synthesized by GeneScript. P2_CCH, P5_CCH, and P5_CCH_A19K were each synthesized under two conditions: standard synthesis using TFA, and the other with TFA-removal using acetate as a counter-ion. A MIC method, adapted for use with cationic antimicrobial peptides, was used to evaluate MIC antimicrobial activity (Hancock 1999).

### Results

MIC and MBC results are provided in the tables below. The units of concentration are μg/mL for MIC and MBC, unless otherwise noted.

TABLE 5
P1_CCH - MIC & MBC Results

| | P1_CCH | |
|---|---|---|
| Peptide | MIC (μg/mL) | MBC (μM/mL) |
| E. coli | >256 | >256 |
| S. aureus | >256 | >256 |
| P. aeruginosa | >256 | >256 |
| S. pyogenes | >256 | >256 |
| M. smegmatis | 256->256 | >256 |

TABLE 6
P2_CCH - MIC & MBC Results

| | P2_CCH | |
|---|---|---|
| Peptide | MIC | MBC |
| E. coli | 256->256 | 256->256 |
| S. aureus | >256 | >256 |
| P. aeruginosa | >256 | >256 |
| S. pyogenes | >256 | >256 |
| M. smegmatis | 16 | 64->256 |

TABLE 7
P3_CCH - MIC & MBC Results

| | P3_CCH | |
|---|---|---|
| Peptide | MIC | MBC |
| E. coli | >256 | >256 |
| S. aureus | >256 | >256 |
| P. aeruginosa | >256 | >256 |
| S. pyogenes | >256 | >256 |
| M. smegmatis | >256 | >256 |

TABLE 8
P4_CCH - MIC & MBC Results

| | P4_CCH | |
|---|---|---|
| Peptide | MIC | MBC |
| E. coli | 128 | 128 |
| S. aureus | >256 | >256 |
| P. aeruginosa | >256 | >256 |
| S. pyogenes | 256 | 256 |
| M. smegmatis | 8 | >256 |

TABLE 9
P5_CCH - MIC & MBC Results

| | P5_CCH | |
|---|---|---|
| Peptide | MIC | MBC |
| E. coli | 16 | 32 |
| S. aureus | 4 | 16 |
| P. aeruginosa | 256->256 | 256->256 |
| S. pyogenes | 128 | 128 |
| M. smegmatis | 2 | >256 |

TABLE 10
P6_CCH - MIC & MBC Results

| | P6_CCH | |
|---|---|---|
| Peptide | MIC | MBC |
| E. coli | 16-64 | 16-64 |
| S. aureus | 128-256 | 256 |
| P. aeruginosa | 256->256 | 256->256 |
| S. pyogenes | 128 | 128 |
| M. smegmatis | 2 | 2->256 |

TABLE 11
P7_CCH - MIC & MBC Results

| | P7_CCH | |
|---|---|---|
| Peptide | MIC | MBC |
| E. coli | >256 | >256 |
| S. aureus | >256 | >256 |
| P. aeruginosa | >256 | >256 |
| S. pyogenes | >256 | >256 |
| M. smegmatis | 32 | >256 |

TABLE 12
P8_CCH - MIC & MBC Results

| | P8_CCH | |
|---|---|---|
| Peptide | MIC | MBC |
| E. coli | >256 | >256 |
| S. aureus | >256 | >256 |
| P. aeruginosa | >256 | >256 |
| S. pyogenes | >256 | >256 |
| M. smegmatis | 128 | 128->256 |

TABLE 13
P9_CCH, P10_CCH, P11_CCH - MIC & MBC Results

| | P9_CCH | | P10_CCH | | P11_CCH | |
|---|---|---|---|---|---|---|
| Peptide | MIC | MBC | MIC | MBC | MIC | MBC |
| E. coli | >256 | >256 | >256 | >256 | >256 | >256 |
| S. aureus | >256 | >256 | >256 | >256 | >256 | >256 |
| P. aeruginosa | >256 | >256 | >256 | >256 | >256 | >256 |
| S. pyogenes | >256 | >256 | >256 | >256 | >256 | >256 |
| M. smegmatis | >256 | >256 | >256 | >256 | >256 | >256 |

TABLE 14
P12_CCH, P13_CCH, P14_CCH - MIC & MBC Results

| | P12_CCH | | P13_CCH | | P14_CCH | |
|---|---|---|---|---|---|---|
| Peptide | MIC | MBC | MIC | MBC | MIC | MBC |
| E. coli | >256 | >256 | >256 | >256 | >256 | >256 |
| S. aureus | >256 | >256 | >256 | >256 | >256 | >256 |
| P. aeruginosa | >256 | >256 | >256 | >256 | >256 | >256 |
| S. pyogenes | >256 | >256 | >256 | >256 | >256 | >256 |
| M. smegmatis | >256 | >256 | >256 | >256 | >256 | >256 |

TABLE 15
P15_CCH and P16_CCH - MIC & MBC Results

| | P15_CCH | | P16_CCH | |
|---|---|---|---|---|
| Peptide | MIC | MBC | MIC | MIC |
| E. coli | >256 | >256 | >256 | >256 |
| S. aureus | >256 | >256 | >256 | >256 |
| P. aeruginosa | >256 | >256 | >256 | >256 |
| S. pyogenes | >256 | >256 | >256 | >256 |
| M. smegmatis | >256 | >256 | >256 | >256 |

TABLE 16
H3, H4, H5 - MIC & MBC Results

| | H3 | | H4 | | H5 | |
|---|---|---|---|---|---|---|
| Peptide | MIC | MBC | MIC | MBC | MIC | MBC |
| E. coli | >256 | >256 | 128 | 128 | >256 | >256 |
| S. aureus | >256 | >256 | 256 | >256 | >256 | >256 |
| P. aeruginosa | >256 | >256 | >256 | >256 | >256 | >256 |
| S. pyogenes | >256 | >256 | >256 | >256 | >256 | >256 |
| M. smegmatis | 8 | >256 | 8 | >256 | 256 | >256 |

TABLE 17
R4 and R4_AcOH - MIC & MBC Results

| | R4 | | R4_AcOH | |
|---|---|---|---|---|
| Peptide | MIC | MBC | MIC | MBC |
| E. coli | 16 | 16 | — | — |
| S. aureus | 4 | 4 | — | — |
| P. aeruginosa | >256 | >256 | >256 | >256 |
| S. pyogenes | — | — | — | — |
| M. smegmatis | 4 | 4 | — | — |

TABLE 18
LL37/Tp - MIC & MBC Results

| | LL37 + ve | | LL37 + ve_AcOH | | Tp-ve | |
|---|---|---|---|---|---|---|
| Peptide | MIC | MBC | MIC | MBC | MIC | MBC |
| E. coli | 64 | 64 | 64 | 64 | >256 | >256 |
| S. aureus | 256 | >256 | 256 | >256 | >256 | >256 |
| P. aeruginosa | 256->256 | 256->256 | >256 | >256 | >256 | >256 |
| S. pyogenes | >256 | >256 | — | — | >256 | >256 |
| M. smegmatis | 4 | 4->256 | 4 | 4 | >256 | >256 |

The following data are presented to show the activity (MIC/MBC in pg/mL) of certain antimicrobial peptides described herein relative to a modified/synthetic version having a specified substitution. While data presented in tables above may be presented again, or may be represented in data provided in the tables below, such duplication is believed to assist the reader in readily noting comparisons. Different Run N umbers noted in the following tables denotes experiments conducted on different days, with each run reflecting multiple n values.

TABLE 19
ATCC E. coli - MIC/MBC Analysis

| | | Run No. | | | | |
|---|---|---|---|---|---|---|
| Peptide | # | 1 | 2 | 3 | 4 | 5 |
| P1_CCH | 1 | NI | | | | |
| P1_CCH_F9R_Y2P | 2 | NI | | | | |
| P2_CCH | 3 | 16/16 | NI | NI | 128/128 | 128/128 |
| P2_CCH_T15K_P7R | 4 | 4/4 | 4/4 | 4/4 | 8/8 | 8/8 |
| P3_CCH | 5 | NI | NI | NI | NI | NI |
| P3_CCH_D8K_Q23C | 6 | 64/64 | 64/64 | 64/64 | 64/64 | 64/64 |
| P4_CCH | 7 | 64/128 | 32/64 | 32/64 | 32/32 | 32/32 |
| P4_CCH_P31K | 8 | 32/32 | 32/32 | 16/32 | 16/32 | 16/32 |
| P5_CCH | 9 | 8/8 | 8/8 | 8/16 | 16/16 | 16/16 |
| P5_CCH_A19K | 10 | 16/16 | 4/4 | 4/4 | 4/4 | 4/4 |
| P6_CCH | 11 | 16/16 | 8/8 | 8/8 | 8/16 | 16/16 |
| P6_CCH_S12K | 12 | 4/4 | 4/4 | 4/4 | 4/4 | 4/4 |
| P7_CCH | 13 | NI | | | | |
| P7_CCH_P9R_R5M | 14 | 256 | | | | |
| P8_CCH | 15 | NI | | | | |
| P8_CCH_N11K | 16 | NI | | | | |
| P11_CCH | 17 | NI | NI | NI | NI | NI |
| P11_CCH_E21K_E23R | 18 | 32/32 | 64 | 64 | 64 | 64 |
| P13_CCH | 19 | NI | NI | NI | NI | NI |
| P13_CCH_N8K_E18K | 20 | 32 | NI | NI | NI | NI |
| HP1 | 21 | 2/2 | 4/4 | 4/4 | 4/4 | 4/4 |
| HP1delta7 | 22 | 8 | 16/16 | 16/16 | 16/16 | 16/16 |
| HP3 | 23 | NI | | | | |
| HP3_S3R | 24 | 128 | | | | |
| HP5 | 25 | NI | | | | |
| HP5_E10K_E14R | 26 | NI | | | | |
| LL37 positive | 27 | 8/8 | 8/8 | | 16/16 | 16/16 |
| P5_Tp Negative | 28 | NI | NI | | NI | NI |

TABLE 20
ESBL E. coli - MIC/MBC Analysis

| | Run No. | | | | |
|---|---|---|---|---|---|
| Peptide | 1 | 2 | 3 | 4 | 5 |
| P1_CCH | NI | | | | |
| P1_CCH_F9R_Y2P | NI | | | | |
| P2_CCH | 16/16 | 16/16 | 16/16 | 64/128 | 128/128 |
| P2_CCH_T15K_P7R | 2/2 | 4/4 | 4/4 | 8/8 | 8/8 |
| P3_CCH | NI | NI | NI | NI | NI |
| P3_CCH_D8K_Q23C | 64/64 | 64/64 | 64/64 | 64/128 | 64/128 |
| P4_CCH | 64/128 | 64/128 | 64/128 | 64/64 | 64/64 |
| P4_CCH_P31K | 32/32 | 32/32 | 32/32 | 32/32 | 32/32 |
| P5_CCH | 8/32 | 8/8 | 8/8 | 16/64 | 32/64 |
| P5_CCH_A19K | 16/16 | 16/16 | 16/16 | 8/8 | 4/4 |
| P6_CCH | 16/64 | 8/32 | 16/32 | 16/16 | 16/16 |
| P6_CCH_S12K | 4/4 | 4/4 | 4/4 | 4/4 | 4/4 |
| P7_CCH | NI | | | | |
| P7_CCH_ P9R_R5M | 128/128 | | | | |
| P8_CCH | NI | | | | |
| P8_CCH_N11K | 128/128 | | | | |
| P11_CCH | NI | NI | NI | NI | NI |
| P11_CCH_E21K_E23R | 32/64 | 16 | 16 | 32/128 | 32/128 |
| P13_CCH | NI | NI | NI | NI | NI |
| P13_CCH_N8K_E18K | NI | NI | NI | NI | NI |
| HP1 | 8/8 | 4/4 | 4/4 | 4/4 | 4/4 |
| HP1delta7 | 32/32 | 32/32 | 32/32 | 64/128 | 64/128 |
| HP3 | NI | | | | |
| HP3_S3R | NI | | | | |
| HP5 | NI | | | | |
| HP5_E10K_E14R | NI | | | | |
| LL37 positive | 8/8 | 4/4 | | 8/8 | |
| P5_Tp Negative | NI | NI | | NI | |

TABLE 21
CPO E. coli (KPC) - MIC/MBC Analysis

| | Run No. | | | | |
|---|---|---|---|---|---|
| Peptide | 1 | 2 | 3 | 4 | 5 |
| P1_CCH | NI | | | | |
| P1_CCH_F9R_Y2P | NI | | | | |
| P2_CCH | 32/32 | 16/16 | 16/16 | 64/64 | 64/64 |
| P2_CCH_T15K_P7R | 8/8 | 8/8 | 8/8 | 8/8 | 8/8 |
| P3_CCH | NI | NI | NI | NI | NI |
| P3_CCH_D8K_Q23C | 64/128 | 128/128 | 128/128 | 64/64 | 64/64 |
| P4_CCH | 32/64 | 32/64 | 32/64 | NI | NI |
| P4_CCH_P31K | 32/32 | 16/32 | 16/32 | 16/32 | 16/32 |
| P5_CCH | 4/4 | 4/4 | 4/4 | 16/16 | 16/16 |
| P5_CCH_A19K | 16/16 | 16/16 | 16/16 | 4/4 | 4/4 |
| P6_CCH | 8/16 | 8/32 | 8/16 | 8/8 | 8/8 |
| P6_CCH_S12K | 4/8 | 4/4 | 4/4 | 4/4 | 4/4 |
| P7_CCH | NI | | | | |
| P7_CCH_P9R_R5M | 256/256 | | | | |
| P8_CCH | NI | | | | |
| P8_CCH_N11K | NI | | | | |
| P11_CCH | NI | NI | NI | NI | NI |
| P11_CCH_E21K_E23R | 64 | 32 | 32 | 32 | 32 |
| P13_CCH | NI | NI | NI | NI | NI |
| P13_CCH_N8K_E18K | 64 | 64 | 64 | NI | NI |
| HP1 | 8/8 | 4/4 | 4/16 | 4/4 | 4/4 |
| HP1delta7 | 16 | 16/32 | 16/64 | 32/32 | 32/32 |
| HP3 | NI | | | | |
| HP3_S3R | 256/256 | | | | |
| HP5 | NI | | | | |
| HP5_E10K_E14R | NI | | | | |
| LL37 positive | 16/16 | 16/16 | | 8/8 | |
| P5_Tp Negative | NI | NI | | NI | |

TABLE 22
CPO E. coli (NDM) - MIC/MBC Analysis

| | Run No. | | | | |
|---|---|---|---|---|---|
| Peptide | 1 | 22 | 3 | 4 | 5 |
| P1_CCH | NI | | | | |
| P1_CCH_F9R_Y2P | NI | | | | |
| P2_CCH | 16/16 | 16/16 | 16/16 | 128/128 | 128/128 |
| P2_CCH_T15K_P7R | 4/4 | 4/4 | 4/4 | 8/8 | 8/8 |
| P3_CCH | NI | NI | NI | NI | NI |
| P3_CCH_D8K_Q23C | 128/128 | 128/128 | 128/128 | 128/128 | NI |
| P4_CCH | 128/256 | 128/128 | 64/128 | 64/64 | 64/64 |
| P4_CCH_P31K | 32 | 32/32 | 32/32 | 64/64 | 32/64 |
| P5_CCH | 8/8 | 4/8 | 8/16 | 32/32 | 32/32 |
| P5_CCH_A19K | 16/16 | 16/16 | 16/16 | 8/8 | 4/4 |
| P6_CCH | 16/16 | 16/16 | 16/16 | 32/64 | 32/64 |
| P6_CCH_S12K | 4/4 | 4/8 | 4/8 | 8/8 | 8/8 |
| P7_CCH | 256 | | | | |
| P7_CCH_P9R_R5M | 256 | | | | |
| P8_CCH | NI | | | | |
| P8_CCH_N11K | 128/128 | | | | |
| P11_CCH | NI | NI | NI | NI | NI |
| P11_CCH_E21K_E23R | 32/128 | 32/128 | 32/128 | 64 | 64 |
| P13_CCH | 256 | NI | NI | NI | NI |
| P13_CCH_N8K_E18K | 128 | 64 | 64 | 64? | 64? |
| HP1 | 8/8 | 8/8 | 8/8 | 8/8 | 4/4 |
| HP1delta7 | 64/128 | 16/32 | 16/32 | 64/64 | 64/64 |
| HP3 | NI | | | | |
| HP3_S3R | 128 | | | | |
| HP5 | NI | | | | |
| HP5_E10K_E14R | NI | | | | |
| LL37 positive | 8/8 | 16/16 | | 8/8 | |
| P5_Tp Negative | 256/256 | NI | | NI | |

TABLE 23
ATCC S. aureus - MIC/MBC Analysis

| | Run No. | | | | |
|---|---|---|---|---|---|
| Peptide | 1 | 2 | 3 | 4 | 5 |
| P1_CCH | NI | | | | |
| P1_CCH_F9R_Y2P | NI | | | | |
| P2_CCH | NI | NI | NI | NI | NI |
| P2_CCH_T15K_P7R | 64/64 | 128/128 | 128/128 | 64/128 | 64/128 |
| P3_CCH | NI | NI | NI | NI | NI |
| P3_CCH_D8K_Q23C | NI | NI | NI | NI | NI |
| P4_CCH | NI | NI | NI | NI | NI |
| P4_CCH_P31K | NI | NI | NI | NI | NI |
| P5_CCH | 4/4 | 8/8 | 8/8 | 8/8 | 8/8 |
| P5_CCH_A19K | 8/8 | 2/2 | 2/2 | 2/2 | 2/2 |
| P6_CCH | 256 | 128 | 128 | NI | NI |
| P6_CCH_S12K | 64/64 | 64/64 | 64/64 | 128/128 | 128/128 |
| P7_CCH | NI | | | | |
| P7_CCH_P9R_R5M | 128 | | | | |
| P8_CCH | NI | | | | |
| P8_CCH_N11K | NI | | | | |
| P11_CCH | 64 | NI | NI | NI | NI |
| P11_CCH_E21K_E23R | 128 | NI | NI | NI | NI |
| P13_CCH | 128 | NI | NI | NI | NI |
| P13_CCH_N8K_E18K | 256 | NI | NI | NI | NI |
| HP1 | 4/8 | 4/4 | 4/4 | 4/4 | 4/4 |
| HP1delta7 | 8/16 | 32/32 | 32/32 | 32/32 | 32/32 |
| HP3 | NI | | | | |
| HP3_S3R | NI | | | | |
| HP5 | NI | | | | |
| HP5_E10K_E14R | NI | | | | |
| LL37 positive | 128 | 128 | | NI | NI |
| P5_Tp Negative | NI | NI | | NI | NI |

TABLE 24
MRSA - MIC/MBC Analysis

| | Run No. | | | | |
|---|---|---|---|---|---|
| Peptide | 1 | 2 | 3 | 4 | 5 |
| P1_CCH | NI | | | | |
| P1_CCH_F9R_Y2P | NI | | | | |
| P2_CCH | NI | NI | NI | NI | NI |
| P2_CCH_T15K_P7R | 64/64 | 32/64 | 64/64 | 32/32 | 32/32 |
| P3_CCH | NI | NI | NI | | |
| P3_CCH_D8K_Q23C | NI | NI | NI | | |
| P4_CCH | NI | NI | NI | | |
| P4_CCH_P31K | NI | NI | NI | | |
| P5_CCH | 2/2 | 2/2 | 4/4 | 8/8 | 4/4 |
| P5_CCH_A19K | 8/8 | 8/8 | 8/8 | 2/2 | 2/2 |
| P6_CCH | 256/256 | 256/256 | 256/256 | 128 | 128 |
| P6_CCH_S12K | 128/128 | 64/64 | 64/128 | 64/64 | 64/64 |
| P7_CCH | NI | | | | |
| P7_CCH_P9R_R5M | NI | | | | |
| P8_CCH | NI | | | | |
| P8_CCH_N11K | NI | | | | |
| P11_CCH | NI | NI | NI | NI | NI |
| P11_CCH_E21K_E23R | NI | NI | NI | NI | NI |
| P13_CCH | NI | NI | NI | NI | NI |
| P13_CCH_N8K_E18K | NI | NI | NI | NI | NI |
| HP1 | 4/4 | 2/2 | 4/4 | 4/8 | 4/8 |
| HP1delta7 | 8 | 8/8 | 8/8 | 16/64 | 16/16 |
| HP3 | NI | | | | |
| HP3_S3R | NI | | | | |
| HP5 | NI | | | | |
| HP5_E10K_E14R | NI | | | | |
| LL37 positive | 256 | NI | | NI | NI |
| P5_Tp Negative | NI | NI | | NI | NI |

TABLE 25
Salmonella Enteritidis - MIC/MBC Analysis

| | Run No. | | | | |
|---|---|---|---|---|---|
| Peptide | 1 | 2 | 3 | 4 | 5 |
| P1_CCH | NI | | | | |
| P1_CCH_F9R_Y2P | NI | | | | |
| P2_CCH | 64/64 | NI | NI | NI | NI |
| P2_CCH_T15K_P7R | 32/32 | 64/64 | 64/64 | 32/128 | 32/128 |
| P3_CCH | NI | NI | NI | NI | NI |
| P3_CCH_D8K_Q23C | NI | NI | NI | NI | NI |
| P4_CCH | NI | NI | NI | NI | NI |
| P4_CCH_P31K | 256/256 | NI | NI | NI | NI |
| P5_CCH | 64/64 | 128/128 | 128/128 | 128/128 | 128/128 |
| P5_CCH_A19K | 64/64 | 16/16 | 32/32 | 16/16 | 16/16 |
| P6_CCH | 128/128 | 64/64 | 64/64 | 64/128 | 128/128 |
| P6_CCH_S12K | 16/16 | 32/32 | 32/32 | 32/32 | 16/32 |
| P7_CCH | NI | | | | |
| P7_CCH P9R R5M | NI | | | | |
| P8_CCH | NI | | | | |
| P8_CCH_N11K | NI | | | | |
| P11_CCH | 32 | 32 | 32 | NI | NI |
| P11_CCH_E21K_E23R | 64 | NI | NI | NI | NI |
| P13_CCH | 16 | 16 | 16 | NI | NI |
| P13_CCH_N8K_E18K | 16 | 16 | 16 | NI | NI |
| HP1 | 32 | 32/32 | 16/16 | 16/16 | 16/16 |
| HP1delta7 | 256 | NI | NI | NI | NI |
| HP3 | NI | | | | |
| HP3_S3R | NI | | | | |
| HP5 | NI | | | | |
| HP5_E10K_E14R | NI | | | | |
| LL37 positive | NI | 128 | | 64/64 | 64/64 |
| P5_Tp Negative | NI | NI | | NI | NI |

TABLE 26
Salmonella Heidelberg (MDR) - MIC/MBC Analysis

| | Run No. | | | | |
|---|---|---|---|---|---|
| Peptide | 1 | 2 | 3 | 4 | 5 |
| P1_CCH | NI | | | | |
| P1_CCH_F9R_Y2P | NI | | | | |
| P2_CCH | 128/128 | NI | NI | NI | NI |
| P2_CCH_T15K_P7R | 16/16 | 64/64 | 32/64 | 64/64 | 32/64 |
| P3_CCH | NI | NI | NI | NI | NI |
| P3_CCH_D8K_Q23C | 256 | NI | NI | NI | NI |
| P4_CCH | 256 | NI | NI | NI | NI |
| P4_CCH_P31K | 128 | 128 | 128 | NI | NI |
| P5_CCH | 32/64 | 128/128 | 128/128 | 128/128 | 128/128 |
| P5_CCH_A19K | 64/64 | 16/16 | 16/16 | 16/16 | 16/16 |
| P6_CCH | 64/256 | 64/64 | 64/64 | 64/64 | 64/64 |
| P6_CCH_S12K | 32/64 | 32/64 | 16/64 | 16/32 | 16/32 |
| P7_CCH | NI | | | | |
| P7_CCH_P9R_R5M | NI | | | | |
| P8_CCH | NI | | | | |
| P8_CCH_N11K | NI | | | | |
| P11_CCH | 128 | NI | NI | NI | NI |
| P11_CCH_E21K_E23R | 128 | NI | NI | NI | NI |
| P13_CCH | 32 | 32? | 32? | NI | NI |
| P13_CCH_N8K_E18K | 32 | 16? | 16? | NI | NI |
| HP1 | 32/32 | 16/16 | 32/32 | 16/16 | 16/16 |
| HP1delta7 | 128 | 128 | 128 | 128 | 128 |
| HP3 | NI | | | | |
| HP3_S3R | NI | | | | |
| HP5 | NI | | | | |
| HP5_E10K_E14R | NI | | | | |
| LL37 positive | NI | NI | | 128 | NI |
| P5_Tp Negative | NI | NI | | | |

Table 27 shows the activity testing of select peptides described herein, for the metrics of Minimum Inhibitory Concentration (MIC) as well as by hemolysis testing to determine HC₅₀ (μg/mL) as an indicator of hemolytic activity based on the concentration of an antimicrobial compound that kills 50% red blood cells using standard methodology. In Table 27, the designations P2, P2M, P3, P3M, P4, P4M, P5, P5M, P6, and P6M refer to peptides P2_CCH, P2_CCH_T5K_P7R, P3_CCH, P3_CCH_D8K_Q23C, P4_CCH, P4_CCH_P31K, P5_CCH, P5_CCH_A19K, P6_CCH, and P6_CCH_S12K, respectively, as defined above.

TABLE 27
Selective Activity Parameters for Antimicrobial Peptides
Activity Test
Minimum Inhibitory Concentration (MIC) (μg/mL)

| | P2// | P3// | P4// | P5// | P6// | | Ran- | LL37 | Tp_P5 |
|---|---|---|---|---|---|---|---|---|---|
| AMP | P2M^{a} | P3M^{a} | P4M^{a} | P5M^{a} | P6M^{a} | Ran-4 | 4M | (+ve) | (−ve) |
| Bacterial Isolate: | Gram negative | | | | | | | | |
| E. coli ATCC 25922 | 16//4-8 | —//64 | 32-64//16-32 | 4-16//4-16 | 8-16//4 | 2-4 | 8-16 | 8-16 | — |
| ESBL E. coli | 16//2-8 | —//64 | 64//32 | 8-32//4-16 | 8-16//4 | 4-8 | 32-64 | 4-8 | — |
| CPO E. coli | 16//4-8 | —//128+ | 64-128// | 4-32//4-16 | 16-32//4-8 | 4-8 | 16-64 | 8-16 | — |
| NDM | | | 32-64 | | | | | | |
| CPO E. coli | 16-32//8 | —//64-128 | 32// | 4-16//4-16 | 8//4 | 4-8 | 16-32 | 8-16 | — |
| KPC | | | 16-32 | | | | | | |
| S. enterica spp. | 64//32-64 | —//— | —//— | 64-128// | 64-128// | 16-32 | — | 64+ | — |
| Enteritidis | | | | 16-64 | 16-32 | | | | |
| S. enterica spp. | 128//16-64 | —//— | —//128+ | 32-128// | 64// | 16-32 | 128 | — | — |
| Heidelberg | | | | 16-64 | 16-32 | | | | |
| Bacterial Isolate: | Gram positive | | | | | | | | |
| S. aureus | —//64-128 | —//— | —//— | 4-8//2-8 | 128+// | 64-128 | 8-23 | 128+ | — |
| ATCC 29213 | | | | | 64-128 | | | | |
| MRSA | —//32-64 | —//— | —//— | 2-8//2-8 | 128+// | 64-128 | 8-16 | — | — |
| | | | | | 64-128 | | | | |

Hemolysis Test HC₅₀ (μg/mL)

| | | | | | | | | | |
|---|---|---|---|---|---|---|---|---|---|
| HC₅₀ (μg/mL)- | —//— | —//— | —//— | 128//— | —//— | 128 | — | — | — |
| 1XPBS | | | | | | | | | |
| HC₅₀ (μg/mL)- | —//64 | —//— | —//— | 8-64// | —//— | 16-64 | — | — | — |
| RPMI | | | | 64-128 | | | | | |

^{a}For data sharing a cell in this table, values are separated by “//”

Table 28 provides an overview of the sequences described herein, and the counterpart SEQ ID NOs. Where “X” is shown in the sequence listing, it is the equal of the three-letter code “Xaa”, meaning that any sequence may be present. Where tables or figures illustrating alignments may show a dash “-”, this indicates an absent residue, in place to maintain the best alignment possible with the comparable sequences shown. Unless otherwise specified, sequences are synthetic and/or derived from Bullfrog Annotated Reference Transcriptome (BART) of the North American bullfrog, Rana (Lithobates) catesbeiana. Synthetic peptides, and/or are isolated sequences.

TABLE 28
Sequences described herein and corresponding SEQ ID NOs

| Table/FIG. | SEQ ID NO |
|---|---|
| Table 1 | SEQ ID NO: 1-14 are provided below. |
| | SEQ ID NO: 1 is: |
| | MFTMKKSLLLLFFLGTISLSLCEQERNADDDQGEVIEQKVKR |
| | SEQ ID NO: 2 is: |
| | AFLSTVKNTLINVAGTMIDIFKCKITGVC |
| | SEQ ID NO: 3 is: |
| | VLLYLIITVSFPRRDANDEDGGEVIKEVVKR |
| | SEQ ID NO: 4 is: |
| | SLSGCWTKSFPRKPCLRNR |
| | SEQ ID NO: 5 is: |
| | MSSFCEITNVALTISLSSPRRGADEEEGNGEKEIKR |
| | SEQ ID NO: 6 is: |
| | SMLSVLKNLGKVGLGFVACKINKQC |
| | SEQ ID NO: 7 is: |
| | MIQSTQKWFKIKYWRVRNRPAMSPDLNPIEHLWRDLKKVVGKR |
| | SEQ ID NO: 8 is: |
| | NPSNLRALEELVKEECSEIPVERCKKLIYGYRK |
| | SEQ ID NO: 9 is: |
| | MRKRMTMRRMMKKKKSEKERRERGKR |
| | SEQ ID NO: 10 is: |
| | MMRVMRRKTKVIWEKKDFIGLYSID |
| | SEQ ID NO: 11 is: |
| | MFFMSSPRRDADEVKEVKR |
| | SEQ ID NO: 12 is: |
| | GFLDIIKNLGKTFAGHMLDKIKCTIGTCPPSP |
| | SEQ ID NO: 13 is: |
| | MITVSSPRRDADGDEGEVEEVKR |
| | SEQ ID NO: 14 is: |
| | GFLDIIKDIGKEFAVKILNNLKCKLAGGCPP |
| FIG. 1 | SEQ ID NO: 15-30 are provided below. |
| | SEQ ID NO: 15 is: |
| | MFTMKKSLLLFFFLGTISLSLCEEERDADDDQGEVVKKEVKR |
| | AFFTIVKNLVINVAGTVIDKMKCKLIGQC |
| | SEQ ID NO: 16 is: |
| | MFTMKKSLLLLFFLGTISLSLCEQERNADDDQGEVIEQKVKR |
| | AFLSTVKNTLINVAGTMIDIFKCKITGVC |
| | SEQ ID NO: 17 is: |
| | MFTLKKSLLLLFFLGTITLSLCEQERGADEEEGNGEKEIKR |
| | SMLSVLKNLGKVGLGFVACKINKQC |
| | SEQ ID NO: 18 is: |
| | MSSFCEITNVALTISLSSPRRGADEEEGNGEKEIKR |
| | SMLSVLKNLGKVGLGFVACKINKQC |
| | SEQ ID NO: 19 is: |
| | MFFMSSPRRDADEVKEVKR |
| | GFLDIIKNLGKTFAGHMLDKIKCTIGTCPPSP |
| | SEQ ID NO: 20 is: |
| | MFTMKKSLLLLFFLGTISLSLCEPQRDADEVKEVKR |
| | GFLDIIKNLGKTFAGHMLDKIKCTIGTCPPSP |
| | SEQ ID NO: 21 is: |
| | MFTLKKSLLLLFFLGTINLSLCEEERDAEEERRDNPDERDVEVEKR |
| | FLPFIARLAAKVFPSIICSVIKKC |
| | SEQ ID NO: 22 is: |
| | MFTMKKSLLLLFFLGTISLSLCEQERNADDDQGEVIEQKVKR |
| | AFLSTVKNTLINVAGTMIDIFKCKITGVC |
| | SEQ ID NO: 23 is: |
| | MFTLKKSLLLLFFLGTITLSLCEQERGADEDNGGEMTEEEVKR |
| | GLFLDTLKGAAKDVAGKLLEGLKCKITGCKP |
| | SEQ ID NO: 24 is: |
| | MFTMKKSLLLLFFLGIISLSLCEQERDANDEEDGGEVTKEVVKR |
| | SLRGCWIKSFPPQPCLGKRLNMN |
| | SEQ ID NO: 25 is: |
| | MFILKKSLLLLFFFGIISLSFCEQERDANDEEDGGEVIKEVVKR |
| | SLRGCWTKSYPPQPCLGKR |
| | SEQ ID NO: 26 is: |
| | VLLYLIITVSFPRRDANDEDGGEVIKEVVKR |
| | SLSGCWTKSFPRKPCLRNR |
| | SEQ ID NO: 27 is: |
| | MFTMKKSEKERRERGKR |
| | MMRVMRRKTKVIWEKKDFIGLYSID |
| | SEQ ID NO: 28 is: |
| | MRKRMTMRRMMKKKKSEKERRERGKR |
| | MMRVMRRKTKVIWEKKDFIGLYSID |
| | SEQ ID NO: 29 is: |
| | MFTMKKSLLLLFFLGTISLSLCEQERDADGDEGEVEEVKR |
| | GFLDIIKDIGKEFAVKILNNLKCKLAGGCPP |
| | SEQ ID NO: 30 is: |
| | MITVSSPRRDADGDEGEVEEVKR |
| | GFLDIIKDIGKEFAVKILNNLKCKLAGGCPP |
| FIG. 4 | SEQ ID NO: 31-34 are provided below. |
| | SEQ ID NO: 31 is: |
| | MGLSATLWFLMGVAAGSMASPLLQWSEDDISVMALYSTDYYNKVS |
| | GEDVLYGLQENNTEYITDEKSRFHQLSFPIQKTVCQKSDNALTDD |
| | CAFKEGGVVKSCTSYFFEEDDRDIIVVICQSQDGHREHSRVRRSR |
| | RGRGGGRRGGSGGRGGRGGGGRSGAGSSIAGVGSRGGGGGRHYA |
| | SEQ ID NO: 32 is: |
| | MGLSATFWFLMGLAASSMASPLLQWSEDDAAVMALYSADHYNKV |
| | SGEDVLYGLLENDTEYITDEKSRFHQLSFPIQETVCQKSDNNAP |
| | TDDCAFKEGGVVKSCTSYFFEEDDRDIVVVNCQSQDSHREHSRV |
| | RRSRSGRGGGGRGGGGRGGSRGGSRSGSRSSIAGGGSRGGSRGG |
| | GTRYA |
| | SEQ ID NO: 33 is: |
| | MIPQLRKWMAIFVMCIVLIHQLEGAPMNSNDGSKTALRLRR |
| | MTPFWRGLSLRPVGASCRDDTECLIRLCRNQRCSLKTFAD |
| | SEQ ID NO: 34 is: |
| | MTPQLRKWTAIFVICIVLIHQLEGAPMSNTAGSKTLLRLRR |
| | MTPFWRGLSLRPVGASCRDDTECLIRLCRKERCSLKTFAD |
| P1 to P16 “natural” | SEQ ID NO: 35-65 are provided below. |
| and “synthetic” | SEQ ID NO: 35 is: FYFPVSRKFGGK |
| example | SEQ ID NO: 36 is: FPFPVSRKRGGK |
| sequences | SEQ ID NO: 37 is: |
| | FFPRVLPLANKFLPTIYCALPKSVGN |
| | SEQ ID NO: 38 is: |
| | FFPRVLRLANKFLPKIYCALPKSVGN |
| | SEQ ID NO: 39 is: |
| | GLLDIIKDIGKITGILMDTLKCQMTGRCPPSS |
| | SEQ ID NO: 40 is: |
| | GLLDIIKKIGKITGILMDTLKCCMTGRCPPSS |
| | SEQ ID NO: 41 is: |
| | GLLDIIKTTGKDFAVKILDNLKCKLAGGCPP |
| | SEQ ID NO: 42 is: |
| | GLLDIIKTTGKDFAVKILDNLKCKLAGGCPK |
| | SEQ ID NO: 43 is: |
| | FFPIIARLAAKVIPSLVCAVTKKC |
| | SEQ ID NO: 44 is: |
| | FFPIIARLAAKVIPSLVCKVTKKC |
| | SEQ ID NO: 45 is: |
| | GLWETIKTTGKSIALNLLDKIKCKIAGGCPP |
| | SEQ ID NO: 46 is: |
| | GLWETIKTTGKKIALNLLDKIKCKIAGGCPP |
| | SEQ ID NO: 47 is: |
| | ATAWRIPPPGMQPIIPIRIRPLCGKQ |
| | SEQ ID NO: 48 is: |
| | ATAWMIPPRGMQPIIPIRIRPLCGKQ |
| | SEQ ID NO: 49 is: FPAIICKVSKNC |
| | SEQ ID NO: 50 is: FPAIICKVSKKC |
| | SEQ ID NO: 51 is: FLTKPGMTFGKLLGK |
| | SEQ ID NO: 52 is: SNRDFFKVNIFRLCG |
| | SEQ ID NO: 53 is: SNRKFFKVRIFRLCG |
| | SEQ ID NO: 54 is: ALVAKIQKFPVFNTLKLCKLELEII |
| | SEQ ID NO: 55 is: ALVAKIQKFPVFNTLKLCKLKLRII |
| | SEQ ID NO: 56 is: IAGQVAAAKQKHI |
| | SEQ ID NO: 57 is: IAGQKARAKQKHI |
| | SEQ ID NO: 58 is: IQRLPVINMLKLWKLELEII |
| | SEQ ID NO: 59 is: IQRLPVIKMLKLWKLELKII |
| | SEQ ID NO: 60 is: IQRLPVIVILPSLYCVICRTC |
| | SEQ ID NO: 61 is: IQRLPVIVILPSLYCVICRKK |
| | SEQ ID NO: 62 is: |
| | LRCPTPHYNFENGIGNHLMWNIIWLNAQQMSYKNK |
| | SEQ ID NO: 63 is: |
| | LRCPTPHYRFENGIGNHLMWNIIWLNAQQMSYCNK |
| | SEQ ID NO: 64 is: SNRDFFMVNIFGLCGPFGIMERKRR |
| | SEQ ID NO: 65 is: SNRKFFMVNIFGLCGPFGIMKRKRR |
| Group P1-Group | SEQ ID NO: 66-166 are provided below |
| P16 variant | XXXPXXXXXGGK (SEQ ID NO: 66) |
| sequences | FYFPXXXXXGGK (SEQ ID NO: 67) |
| | XYFXXSRKXXXX (SEQ ID NO: 68) |
| | FXFXVSRKXXXX (SEQ ID NO: 69) |
| | XYFXXXXKFXXK (SEQ ID NO: 70) |
| | XXXXXXXXFGGK (SEQ ID NO: 71) |
| | FYXPVXRXFXXX (SEQ ID NO: 72) |
| | FFPRXXXXXXXFLPTXXXXXXXSVGN (SEQ ID NO: 73) |
| | XXXXVLPLANKXXXXIYCXXXXXXXX (SEQ ID NO: 74) |
| | FFPXXLPLANXXLPXXXXXLPXXVGN (SEQ ID NO: 75) |
| | XXPRVLXXXXXXXXXXXXXXPKSXXX (SEQ ID NO: 76) |
| | FXXXXXXLANKXXXTIYCXXXXSVXX (SEQ ID NO: 77) |
| | FFXXVLPXXXXXLXTXYCALPKXVXN (SEQ ID NO: 78) |
| | XXXXXXXLANXFXPXIXXALPKXXGX (SEQ ID NO: 79) |
| | GLLXXXXXXXXXXXXXXXXXXXXXXXXCPPSS (SEQ ID NO: 80) |
| | XXXXXXKXXXKXXGXLMXXXXXXMXGXXPPXS (SEQ ID NO: 81) |
| | GLLXIIXXXGXTTGILMXXLXXXMXGXXPPXX (SEQ ID NO: 82) |
| | XLXXXXXXXXKXXXXXXXTLKCQMTXXCXXSS (SEQ ID NO: 83) |
| | GLLXIIKXIGKITGILMXXLKXXXXGXXXXXX (SEQ ID NO: 84) |
| | GXLXIIKXTGXXIXIXMXTLKCQXTGRXPPSS (SEQ ID NO: 85) |
| | GLLXXXKXIGKXIXIXXXILKXQXTGRXXXXX (SEQ ID NO: 86) |
| | XXXXIIXXTXXIXGXLXXTXXCQXTXRXXXXX (SEQ ID NO: 87) |
| | GLLXIIXXXGXXXXXXILXXLXXXLAGGXXX (SEQ ID NO: 88) |
| | GLLXXXKTIGKXFAVKILXNLXXXXXXXXPP (SEQ ID NO: 89) |
| | XXXXIIKTXXXXFAVXXXXXXKCKLAGGXXX (SEQ ID NO: 90) |
| | XXXXIIXXXXKXXXXKXXXNLKCKXXXXCPP (SEQ ID NO: 91) |
| | GLLXXXKTTGXXXXXXXLXNLKCXXAXXCXX (SEQ ID NO: 92) |
| | GLLXXXKXXXXXFAVXXLXXLXXXXXXXXPP (SEQ ID NO: 93) |
| | XXXXXXXLAAKXXXSLVXXXXKKC (SEQ ID NO: 94) |
| | XFPIIAXLAAXVIPXLVXAVTXXX (SEQ ID NO: 95) |
| | FFPXXAXXXXKXXPXXXXXXXXXX (SEQ ID NO: 96) |
| | XXXXXXXLAAXVIPXLXXXXTXXX (SEQ ID NO: 97) |
| | FFPIIAXXXXXXXXXXVCAVTKKC (SEQ ID NO: 98) |
| | XXXIIXRXXXKVIXSXVCXVTKKC (SEQ ID NO: 99) |
| | FFPIIARLAAXVIXSLXCAVXXXX (SEQ ID NO: 100) |
| | GLWETIKXXXKXXXXXXXXKXXXXXXGGCPP (SEQ ID NO: 101) |
| | XXXXXXXTTGXXXXXXXXXXXKCKXXXXCXX (SEQ ID NO: 102) |
| | XXXXTIXXXGXXIALXLLXXIXXXIAXXXPP (SEQ ID NO: 103) |
| | GLWETXKITXXSXXLNLLDKIXXKIAXXXPP (SEQ ID NO: 104) |
| | XXXXXIKXXGKSIALXXXXKXKXKXXGGXXX (SEQ ID NO: 105) |
| | XXXXXXXXXXKSIAXNLLXXIXCXIAGGXXX (SEQ ID NO: 106) |
| | ATAWXIXXXGMXXIIXIXIXXLXGXX (SEQ ID NO: 107) |
| | XXXWXIXXXGMQXXXXXXXXXXCGKQ (SEQ ID NO: 108) |
| | XXXXXXPPPXXQPXXPXXXXPXXXXX (SEQ ID NO: 109) |
| | ATAXXXPPPXXXPXXPXXXXPXCXKQ (SEQ ID NO: 110) |
| | XXXWXIXXXGMXXXXXIXIXXLXGXX (SEQ ID NO: 111) |
| | XXXXXXXXXXXXPXXPXXIXXLXGXX (SEQ ID NO: 112) |
| | ATAXRXXXXXXQXIIXIRIRXLCXKQ (SEQ ID NO: 113) |
| | FPAIIXXXXXXX (SEQ ID NO: 114) |
| | FXXXXCXXXKXC (SEQ ID NO: 115) |
| | XXAIIXXVSKXX (SEQ ID NO: 116) |
| | XPAXXCKXXXXX (SEQ ID NO: 117) |
| | FPXXXXXVSKNC (SEQ ID NO: 118) |
| | XXXIICKVSXNX (SEQ ID NO: 119) |
| | FLTFXGXXFGXXXGX (SEQ ID NO: 120) |
| | XXXXPGMXFXXLLXX (SEQ ID NO: 121) |
| | XXXXPGMXXXKXXXK (SEQ ID NO: 122) |
| | FLTFXXXXXXXLLGX (SEQ ID NO: 123) |
| | FXXFXXXTFGKXXXK (SEQ ID NO: 124) |
| | XLTXXXXTFGKLLGK (SEQ ID NO: 125) |
| | XXXXFFXVNIFXLXX (SEQ ID NO: 126) |
| | SNXXXXXVXXXRXXX (SEQ ID NO: 127) |
| | XXXXFFXXXIFXLCG (SEQ ID NO: 128) |
| | SNXXXXKXXXXXLCG (SEQ ID NO: 129) |
| | XXRXXXKVNIFXXCX (SEQ ID NO: 130) |
| | SXXXFFXVXIXXXXG (SEQ ID NO: 131) |
| | SXRDFFKXNXXRXCX (SEQ ID NO: 132) |
| | XXXXXIQKXXXXNTLKXXKXXLXXX (SEQ ID NO: 133) |
| | ALVAKXXXFPVFXXXXLCXLXXXXX (SEQ ID NO: 134) |
| | ALVAKIQKXXXXXXXXXXKLXXXII (SEQ ID NO: 135) |
| | XXXXXXXKXPXXNTLKXCKXEXEXX (SEQ ID NO: 136) |
| | XXXXXXQXFXVFXTLKLXKLXLXXX (SEQ ID NO: 137) |
| | XLVAKIXXXPVXNXXXLXXXXLXII (SEQ ID NO: 138) |
| | AXXAXIXXFXXFXXXXXCXXEXEII (SEQ ID NO: 139) |
| | XXGQVXXXKXKXX (SEQ ID NO: 140) |
| | IAXXXAAAXXXXX (SEQ ID NO: 141) |
| | XXGXXXXXKXKHI (SEQ ID NO: 142) |
| | IXXQVXXXKQKHI (SEQ ID NO: 143) |
| | XXXQVXXXXQXHI (SEQ ID NO: 144) |
| | XXRXPXXXXXKLWKXXLXXX (SEQ ID NO: 145) |
| | IQXXXVXXXLXXXXLXXXII (SEQ ID NO: 146) |
| | XXXLXXXNMXXXWKXXXXXX (SEQ ID NO: 147) |
| | XXXLPXINMXKLXXXXLXXX (SEQ ID NO: 148) |
| | IQRLXXXXXXXSLYXXXCRTC (SEQ ID NO: 149) |
| | XXXLPVXVXLPSLYXXXXXXX (SEQ ID NO: 150) |
| | IQRXXXIVIXXXXXCVIXXXX (SEQ ID NO: 151) |
| | XXXXPVXXXXPSLYXXXCRIC (SEQ ID NO: 152) |
| | IQRLXVIXILXXXXCXXCXXC (SEQ ID NO: 153) |
| | XXXLPXXXXXPXXXXVIXXTX (SEQ ID NO: 154) |
| | XXXLXVIVILXSLYCVICRTC (SEQ ID NO: 155) |
| | LXXPXPXYXFXXGIGXXXXWXXXWLNAQQMXXXXX |
| | (SEQ ID NO: 156) |
| | XXCPTPXXXFXXXXXNHLXXXIIWLXXXXMXXXXX |
| | (SEQ ID NO: 157) |
| | LRCXXXXXXXENGXXXXXMWNXXXXXXXXXSYKNK |
| | (SEQ ID NO: 158) |
| | XXXXTXHYNXENGIGNHLMXNXXXXNXQXXXXKXK |
| | (SEQ ID NO: 159) |
| | LXCXXXHXNXXXXXGNHXXWXXXWLXXXXMSXXNX |
| | (SEQ ID NO: 160) |
| | XRXPXPHXXFXXXXXXXXXXXIIXXXAXQXSYXXX |
| | (SEQ ID NO: 161) |
| | SNRXXXMXXXXGLXGPXXIMXXXXX (SEQ ID NO: 162) |
| | XXXXFFMXXXXXXCXXFGXXXXKXX (SEQ ID NO: 163) |
| | SNRDXXXXXIFGLXGPXXIMXRKRR (SEQ ID NO: 164) |
| | SXXXXXXVNXXXXCXXFGXXEXXXX (SEQ ID NO: 165) |
| | XXXXXXXVNIFXXXXPXXXXXRXRR (SEQ ID NO: 166) |
| Other Putative | KSKLSLKKQGTIHLDAQSSCDVMHFPKCDLAPNVQRQAWLFKVA |
| AMPs | SKEAKELRYYLLNPYLDVSARNVGSKV (SEQ ID NO: 167) |
| | KAGEGERGEREVLNHQKTILEPSSCPLISPHSTGLGHRPSLFRL |
| | TLA (SEQ ID NO: 168) |
| | LKGIKNAAQLLRFPPNCKLCSCIVFVHKDHCVVQEASGVERF |
| | (SEQ ID NO: 169) |
| | NAARDHSATRCKQRSARLQIAAQDYRSQRSARLQIATQRKITD |
| | RNTA |
| | (SEQ ID NO: 170) |
| | LKPSNIQVKLQYIYW (SEQ ID NO: 171) |
| Further Putative | MNCGSFPCDACDVCEYVDAKTKLKLPNGRWHSIQFRVICQTPG |
| AMP Precursors | VIYLAQCLCGGFYIGKTKRQFFKRIRDHIKPIRKNKMDTAISR |
| | HVGIHHNFNPQFIKFSALEHIPQTLAVAALIASCYN |
| | (SEQ ID NO: 172) |
| | MEEIVFPLQHPFHLDCLFFLLRHLSWEKT (SEQ ID NO: 173) |
| | MSIKKKEEMIQVKGMLKWKNDFYQLLERFSVLCLEKNPEMLKL |
| | (SEQ ID NO: 174) |
| | MIQVKGMLKWKNDFYQLLERFSVLCLEKNPEMLKL |
| | (SEQ ID NO: 175) |
| | MPKKKEETIQMKGMLKWKNDFFQLLHA (SEQ ID NO: 176) |
| | MSGSRIGLPLALFPVTFVKISLFILLSSSSSAFLLGEHSYC |
| | (SEQ ID NO: 177) |
| | MSSPRRDANEEERRDDPDERDVEVEKRLLPVITSENVLV |
| | HRGGQKAGMDHREVTQGWREDLGHQEELSLNLQENNGGH |
| | PQFMPFQ (SEQ ID NO: 178) |

In the preceding description, for purposes of explanation, numerous details are set forth in order to provide a thorough understanding of the embodiments. However, it will be apparent to one skilled in the art that these specific details are not required.

The above-described embodiments are intended to be examples only. A Iterations, modifications and variations can be effected to the particular embodiments by those of skill in the art. The scope of the claims should not be limited by the particular embodiments set forth herein, but should be construed in a manner consistent with the specification as a whole.

### REFERENCES

The following publications are incorporated by reference herein.
1. Munita, J. M. & Arias, C. A. Mechanisms of Antibiotic Resistance. in Virulence Mechanisms of Bacterial Pathogens, Fifth Edition (eds. Kudva, I. T. et al.) 481-511 (American Society of Microbiology, 2016). doi:10.1128/microbiolspec.VMBF-0016-2015
2. Nathan, C. & Cars, O. Antibiotic Resistance—Problems, Progress, and Prospects. N. Engl. J. Med. 371, 1761-1763 (2014).
3. Antimicrobial resistance: global report on surveillance. (World Health Organization, 2014).
4. World Health Organization. Global action plan on antimicrobial resistance. (2015).
5. Jantaruk, P., Roytrakul, S., Sitthisak, S. & Kunthalert, D. Potential role of an antimicrobial peptide, KLK in inhibiting lipopolysaccharide-induced macrophage inflammation. PLOS ONE 12, e0183852 (2017).
6. Bahar, A. & Ren, D. Antimicrobial Peptides. Pharmaceuticals 6,1543-1575 (2013).
7. Brandenburg, K., Heinbockel, L., Correa, W. & Lohner, K. Peptides with dual mode of action: Killing bacteria and preventing endotoxin-induced sepsis. Biochim. Biophys. Acta BBA—Biomembr. 1858, 971-979 (2016).
8. Lehrer (2013). “Innate Immunity in Plants: The Role of Antimicrobial Peptides” In: Antimicrobial peptides and innate immunity. Springer (Basel, Switzerland); Hiemstra & Zaat (Editors.) Ch. 2, p. 29-51.
9. Conlon, J. M. & Mechkarska, M. Host-defense peptides with therapeutic potential from skin secretions of frogs from the family Pipidae. Pharmaceuticals 7, 58-77 (2014).
10. Waghu, F. H., Barai, R. S., Gurung, P. & Idicula-Thomas, S. CAMP_{R3}: a database on sequences, structures and signatures of antimicrobial peptides: Table 1. Nucleic Acids Res. 44, D1094-D1097 (2016).
11. Andersson, D. I., Hughes, D. & Kubicek-Sutherland, J. Z. Mechanisms and consequences of bacterial resistance to antimicrobial peptides. Drug Resist. Updat. 26, 43-57 (2016).
12. Beckloff, N. & Diamond, G. Computational analysis suggests beta-defensins are processed to mature peptides by signal peptidase. Protein Pept. Lett. 15, 536-540 (2008).
13. Aittomäki, S. et al. Proprotein convertase Furin1 expression in the Drosophila fat body is essential for a normal antimicrobial peptide response and bacterial host defense. FASEB J. fj.201700296R (2017).
14. Joo, H.-S., Fu, C.-I. & Otto, M. Bacterial strategies of resistance to antimicrobial peptides. Philos. Trans. R. Soc. B Biol. Sci. 371, 20150292 (2016).
15. Valore, E. V. & Ganz, T. Posttranslational processing of hepcidin in human hepatocytes is mediated by the prohormone convertase furin. Blood Cells. Mol. Dis. 40, 132-138 (2008).
16. Nguyen, L. T., Haney, E. F. & Vogel, H. J. The expanding scope of antimicrobial peptide structures and their modes of action. Trends Biotechnol. 29, 464-472 (2011).
17. Haney, E. F. et al. Mechanism of action of puroindoline derived tryptophan-rich antimicrobial peptides. Biochim. Biophys. Acta BBA—Biomembr. 1828, 1802-1813 (2013).
18. Otvos J r., L. Immunomodulatory effects of anti-microbial peptides. Acta Microbiol. Immunol. Hung. 63, 257-277 (2016).
19. Schadich, E., Cole, A. L. J., Squire, M. & M ason, D. Skin peptides of different life stages ofEwing's tree frog. J. Exp. Zool. Part Ecol. Genet. Physiol. 313A, 532-537 (2009).
20. Batista, C. V., et al. A novel heterodimeric antimicrobial peptide from the tree-frog Phyllomedusa distincta. FEBS Lett. 494, 85-89 (2001).
21. Ge, L. et al. Balteatide: A novel antimicrobial decapeptide from the skin secretion of the purple-sided leaf frog, Phyllomedusa baltea. Sci. World J. 2014, 1-8 (2014).
22. Luca, V., Stringaro, A., Colone, M., Pini, A. & Mangoni, M. L. Esculentin (1-21), an amphibian skin membrane-active peptide with potent activity on both planktonic and biofilm cells of the bacterial pathogen Pseudomonas aeruginosa. Cell. Mol. Life Sci. 70, 2773-2786 (2013).
23. Liang, T. et al. Molecular cloning and expression analysis of liver-expressed antimicrobial peptide 1 (LEAP-1) and LEAP-2 genes in the blunt snout bream (Megalobrama amblycephala). Fish Shellfish Immunol. 35, 553-563 (2013).
24. Krause, A. Isolation and biochemical characterization of LEAP-2, a novel blood peptide expressed in the liver. Protein Sci. 12, 143-152 (2003).
25. Zhang, S. et al. Evolution, expression, and characterisation of liver-expressed antimicrobial peptide genes in ancient chondrostean sturgeons. Fish Shellfish Immunol. (2018).
26. Calhoun, D. M. et al. Role of antimicrobial peptides in amphibian defense against trematode infection. EcoHealth 13, 383-391 (2016).
27. Woodhams, D. C. et al. Life history linked to immune investment in developing amphibians. Conserv. Physiol. 4, cow025 (2016).
28. Hammond, S. A. et al. The North American bullfrog draft genome provides insight into hormonal regulation of long noncoding RNA. Nat. Commun. 8, (2017).
29. Hancock, R. E. W. Modified MIC method for cationic antimicrobial peptides. (1999). Available at: http://cmdr.ubc.ca/bobh/method/modified-mic-method-for-cationic-antimicrobial-peptides/. (Accessed: 22 Sep. 2017)
30. Dürr, U. H. N., Sudheendra, U. S. & Ramamoorthy, A. LL-37, the only human member of the cathelicidin family of antimicrobial peptides. Biochim. Biophys. Acta BBA—Biomembr. 1758, 1408-1425 (2006).
31. Goraya, J., K noop, F. C. & Conlon, J. M. Ranatuerins: antimicrobial peptides isolated from theskin of theAmerican bullfrog, Rana catesbeiana. Biochem Biophys Res Commun 250, 589-592 (1998).
32. [This numeral intentionally left blank].
33. Jackman, K. W. et al. Transcriptomics investigation of thyroid hormone disruption in the olfactory system of the Rana [Lithobates] catesbeiana tadpole. Aquat. Toxicol. 202, 46-56 (2018).
34. Reilly, D. S., Tomassini, N. & Zasloff, M. Expression of magainin antimicrobial peptide genes in the developing granular glands of Xenopus skin and induction by thyroid hormone. Dev. Biol. 162, 123-133 (1994).
35. Clark, D. P., Durell, S., Maloy, W. L. & Zasloff, M. Ranalexin. A novel antimicrobial peptide from bullfrog (Rana catesbeiana) skin, structurally related to the bacterial antibiotic, polymyxin. J. Biol. Chem. 269, 10849-10855 (1994).
36. Katzenback, B. A. et al. Regulation of the Rana sylvatica brevinin-1SY antimicrobial peptide during development and in dorsal and ventral skin in response to freezing, anoxia and dehydration. J. Exp. Biol. 217, 1392-1401 (2014).
37. Ohnuma, A., Conlon, J. M. & Iwamuro, S. Developmental and thyroid hormone-induced expression of preprotemporin genes in the skin of J apanese mountain brown frog Rana ornativentris. Ann. N. Y. Acad. Sci. 1163, 494-496 (2009).
38. Zaiou, M. & Gallo, R. L. Cathelicidins, essential gene-encoded mammalian antibiotics. J. Mol. Med. 80, 549-561 (2002).
39. Conlon, J. M. et al. Host defense peptides from Lithobates forreri, Hylarana luctuosa, and Hylarana signata (Ranidae): Phylogenetic relationships inferred from primary structures of ranatuerin-2 and brevinin-2 peptides. Comp. Biochem. Physiol. PartD Genomics Proteomics 9, 49-57 (2014).
40. Kościuczuk, E. M. et al. Cathelicidins: family of antimicrobial peptides. A review. Mol. Biol. Rep. 39, 10957-10970 (2012).
41. Zhang, S. et al. Evolution, expression, and characterisation of liver-expressed antimicrobial peptide genes in ancient chondrostean sturgeons. Fish Shellfish Immunol. 79, 363-369 (2018).
42. Conlon, J. M., K olodziejek, J. & Nowotny, N. Antimicrobial peptides from the skins of North American frogs. Biochim. Biophys. Acta BBA—Biomembr. 1788, 1556-1563 (2009).
43. Unubol, N. et al. Peptide Antibiotics developed by mimicking natural antimicrobial peptides. Clin. Microbiol. Open Access 06, (2017).
44. Rollins-Smith, L. A. The role of amphibian antimicrobial peptides in protection of amphibians from pathogens linked to global amphibian declines. Biochim. Biophys. Acta BBA—Biomembr. 1788, 1593-1599 (2009).
45. Rollins-Smith, L. A. Amphibian immunity-stress, disease, and climate change. Dev. Comp. Immunol. 66, 111-119 (2017).
46. Holden, W. M., Reinert, L. K., Hanlon, S. M., Parris, M. J. & Rollins-Smith, L. A. Development of antimicrobial peptide defenses of southern leopard frogs, Rana sphenocephala, against the pathogenic chytrid fungus, Batrachochytrium dendrobatidis. Dev. Comp. Immunol. 48, 65-75 (2015).
47. Conlon, J. M. et al. Evaluation of the skin peptide defenses of the Oregon spotted frog Rana pretiosa against infection by the chytrid fungus Batrachochytrium dendrobatidis. J Chem Ecol 39, 797-805 (2013).
48. Robertson, G. et al. De novo assembly and analysis of RNA-seq data. Nat. Methods 7, 909-912 (2010).
49. Mistry, J., Finn, R. D., Eddy, S. R., Bateman, A. & Punta, M. Challenges in homology search: HMMER3 and convergent evolution of coiled-coil regions. Nucleic Acids Res. 41, e121-e121 (2013).
50. Jones, P. et al. InterProScan 5: genome-scale protein function classification. Bioinformatics 30, 1236-1240 (2014).
51. Finn, R. D. et al. The Pfam protein families database: towards a more sustainable future. Nucleic Acids Res. 44, D279-D285 (2016).
52. Wernersson, R. Virtual Ribosome—a comprehensive translation tool with support for sequence feature integration. Nucl. Acids Res. 34, W385-W388 (2006).
53. Sievers, F. et al. Fast, scalable generation of high-quality protein multiple sequence alignments using Clustal Omega. Mol. Syst. Biol. 7, 539-539 (2014).
54. Wu, T. D. & Watanabe, C. K. GMAP: a genomic mapping and alignment program for mRNA and EST sequences. Bioinformatics 21, 1859-1875 (2005).
55. Cameron, C. E., Brouwer, N. L., Tisch, L. M. & Kuroiwa, J. M. Y. Defining the Interaction of the Treponema pallidum Adhesin Tp0751 with Laminin. Infect. Immun. 73, 7485-7494 (2005).
56. Cockerill, F. & Clinical and Laboratory Standards Institute. Methods for dilution antimicrobial susceptibility tests for bacteria that grow aerobically: approved standard. (Clinical and Laboratory Standards Institute, 2015).

1. An antimicrobial peptide consisting of the amino acid sequence according to any one of SEQ ID NOs:1-45 and 47-178.

2. A composition comprising the antimicrobial peptide according to claim 1, and a pharmaceutically acceptable excipient.

3. The composition of claim 2, wherein the composition is formulated for oral, injectable, rectal, topical, transdermal, nasal, or ocular delivery.

4. The composition of claim 2, wherein the composition is lyophilized.

5. A lipid vesicle comprising the antimicrobial peptide of claim 1.

6. A nucleic acid molecule encoding the antimicrobial peptide of claim 1.

7. A vector comprising the nucleic acid molecule of claim 6.

8. A kit for identifying a target molecule associated with an infectious agent, said kit comprising the antimicrobial peptide of claim 1 together with instructions for conducting a method of identifying a target molecule associated with an infectious agent, wherein said target molecule binds to the antimicrobial peptide,
said method comprising the step of screening a library of candidate target molecules associated with the infectious agent, for a molecule that binds to the antimicrobial peptide;
wherein said infectious agent is Gram-negative bacteria, Gram-positive bacteria, acid fast bacteria, or a drug resistant bacteria.

9. A kit for identifying a target molecule for modulating biological activity, said kit comprising the antimicrobial peptide of claim 1 together with instructions for conducting a method of identifying a target molecule for modulating biological activity, wherein said target molecule binds to the antimicrobial peptide,
said method comprising the step of screening a library of candidate target molecules for a molecule that binds to the antimicrobial peptide.

10. A method of treating bacterial infection in a subject in need thereof, wherein the method comprises administering to the subject a therapeutically effective amount of the antimicrobial peptide according to claim 1.

11. The method of claim 10, wherein the bacteria is Gram-negative bacteria.

12. The method of claim 10, wherein the bacteria is Gram-positive bacteria.

13. The method of claim 10, wherein the bacteria is Escherichia coli, Salmonella enterica, Staphylococcus aureus, Pseudomonas aeruginosa, Streptococcus pyogenes, Mycobacterium smegmatis, Methicillin-resistant Staphylococcus aureus (MRSA), Salmonella enteritidis, or Salmonella Heidelberg.
